(19) United States
(12) Patent Application Publication
(10) Pub. No.: US 2024/0424157 A1
(43) Pub. Date: Dec. 26, 2024

(54) UV STERILIZATION SYSTEM AND DEVICE AND RELATED METHODS

(71) Applicant: SEAL SHIELD, LLC, JACKSONVILLE, FL (US)

(72) Inventors: CHRISTIAN DAVIS, JACKSONVILLE, FL (US); NORMAN RANDALL HORN, JACKSONVILLE, FL (US); NADUN KURUPPUMULLAGE, JACKSONVILLE, FL (US); BRADLEY WHITCHURCH, JACKSONVILLE, FL (US)

(21) Appl. No.: 18/822,790

(22) Filed: Sep. 3, 2024

Related U.S. Application Data
(63) Continuation-in-part of application No. 17/654,253, filed on Mar. 10, 2022, now Pat. No. 12,076,455.
Application No. 17/654,253 is a continuation of application No. 16/846,550, filed on Apr. 13, 2020, now Pat. No. 11,305,027.
Application No. 16/846,550 is a division of application No. 15/898,930, filed on Feb. 19, 2018, now Pat. No. 11,058,783.
(60) Provisional application No. 62/460,347, filed on Feb. 17, 2017.
(60) Provisional application No. 62/460,516, filed on Feb. 17, 2017.

(51) Int. Cl. A61L 2/10; A61L 2/24; G06K 7/10

(52) U.S. Cl. CPC A61L 2/10; A61L 2/24; G06K 7/10366; A61L 2202/11; A61L 2202/122; A61L 2202/14

(57) ABSTRACT

A UV sterilization device may include a housing defining a cavity, and having a door configured to permit access to the cavity, a tray carried within the cavity and configured to receive a device, and a UV CBA carried within the cavity adjacent the tray. The UV CBA may include a circuit board, and LED UV sources carried by the circuit board and configured to irradiate the device with UV radiation with an emission spectrum having a spectral width less than the entire UV band. The tray may include a material transparent to the emission spectrum. The UV sterilization device may also include a controller coupled to the UV CBA and configured to selectively power the UV CBA for disinfecting the device. In particular, the LED UV sources may be configured to perform 360 degree irradiation of the device without UV shadowing.

### RELATED APPLICATION

This application is a continuation-in-part of application Ser. No. 17/654,253 filed Mar. 10, 2022, now U.S. Pat. No. 12,076,455, which is a continuation of application Ser. No. 16/846,550 filed Apr. 13, 2020, which is a divisional of application Ser. No. 15/898,930 filed Feb. 19, 2018, now U.S. Pat. No. 11,058,783, which claims priority to application Ser. No. 62/460,516 filed Feb. 17, 2017, and application Ser. No. 62/460,347 filed Feb. 17, 2017, the entire subject matter of these applications is incorporated herein by reference in its entirety.

### TECHNICAL FIELD

The present disclosure relates to the field of medical devices, and, more particularly, to sterilization device and related methods.

### BACKGROUND

Health care facilities, such as hospitals, are under increasing financial pressure due to the global economic downturn, the determination of the federal and state payers to reduce health care payments, and the trend of private insurers to move their insured to high deductible plans. Also, government payment strategies now include reductions in payments for failures to achieve certain outcome and quality targets, a move that private insurers are sure to follow.

Infection controls is an increasingly important aspect of quality at health care facilities. Several approaches to maintaining the sterile field and sterilizing anything that comes in to contact with patient have been disclosed. For example, health care facilities have instituted rigorous hand washing and sterilization procedures, such as requiring hand sanitizer and hand washing before and after interacting with a patient.

As mobile device technology has permeated every aspect of society, in most health care facilities, many personnel carry one or more mobile devices. For example, a user may carry a typical voice communications handset for voice calls, and a tablet/laptop computing device for accessing patient records wirelessly. Of course, since these devices go everywhere the user goes, they accumulate biological contaminants and must be sterilized from time to time.

### SUMMARY

Generally, an ultraviolet (UV) sterilization device may include a housing defining a cavity therein, and having a door configured to permit access to the cavity, at least one tray carried within the cavity and configured to receive at least one device, and at least one UV circuit board assembly (CBA) carried within the cavity adjacent the at least one tray. The at least one UV CBA may include a circuit board, and a plurality of light emitting diode ultraviolet (LED UV) sources carried by the circuit board and configured to irradiate the at least one device with UV radiation with an emission spectrum having a spectral width less than the entire UV band. The at least one tray comprising a material transparent to the emission spectrum. The UV sterilization device may also include a controller coupled to the at least one UV CBA and configured to selectively power the at least one UV CBA for disinfecting the at least one device. In particular, the plurality of LED UV sources may be configured to perform 360 degree irradiation of the at least one device without UV shadowing.

In some embodiments, the UV sterilization device may also include a positive air pressure source carried by the housing and configured to create positive air pressure in the cavity when the door is open, and an air filter coupled to the positive air pressure source. Also, the controller may be configured to drive the plurality of LED UV sources with a stepped waveform. The controller may be configured to monitor duty cycle of the plurality of LED UV sources. The material may comprise quartz, for example.

Furthermore, the controller may be configured to selectively power the at least one UV CBA when the at least one device is detected on a respective tray. The UV sterilization device may also include a keypad carried on an external surface of the housing and coupled to the controller, and the controller may be configured to unlock the door based upon a code received from the keypad. The UV sterilization device may further include a transceiver configured to communicate with a server, and the transceiver may comprise a wireless transceiver coupled to the controller and configured to communicate with the server via a wireless base station. The UV sterilization device may have a near field communications (NFC) device configured to communicate with the at least one device when external to the cavity and unlock the door when the at least one device is authorized.

Another aspect is directed to a method of making an UV sterilization device. The method may include forming a housing defining a cavity therein, and having a door configured to permit access to the cavity, and positioning at least one tray to be carried within the cavity and configured to receive at least one device. The method may also include positioning at least one UV CBA to be carried within the cavity adjacent the at least one tray and comprising a circuit board, and a plurality of LED UV sources to be carried by the circuit board and configured to irradiate the at least one device with UV radiation with an emission spectrum having a spectral width less than the entire UV band, the at least one tray comprising a material transparent to the emission spectrum. The method may further comprise coupling a controller to the at least one UV CBA, the controller configured to selectively power the at least one UV CBA for disinfecting the at least one device.

### BRIEF DESCRIPTION OF THE DRAWINGS

FIGS. 1-3 are schematic diagrams of varying embodiments of the UV sterilization system, according to the present disclosure.

FIGS. 4-5 are diagrams of illustrating structure of the UV sterilization system, according to the present disclosure.

FIGS. 6 and 7 are perspective front and back views of a UV sterilization device, according to the present disclosure.

FIG. 8 is a front elevational view of the UV sterilization device of FIGS. 6-7.

FIG. 9 is a partial front perspective view of the UV sterilization device of FIGS. 6-7 with the door removed.

FIG. 10 is a section view of the UV sterilization device of FIGS. 6-7.

FIGS. 11-13 are perspective and top plan elevational views of UB circuit boards from the UV sterilization device of FIGS. 6-7.

FIG. 14 is a perspective view of a UV CBA from the UV sterilization device of FIGS. 6-7.

FIG. 15 is a section view of a light louver from the UV sterilization device of FIGS. 6-7.

FIG. 16 is a partial front elevational view of the UV sterilization device of FIGS. 6-7.

FIG. 17 is a back plant elevational view of the UV sterilization device of FIGS. 6-7.

FIGS. 18-19 are partial front perspective views of the UV sterilization device of FIGS. 6-7.

FIGS. 20A-20B are top plan elevational views of UB circuit boards from the UV sterilization device of FIGS. 6-7.

FIGS. 21-22 are a flowchart for operation of the UV sterilization device of FIGS. 6-7.

FIGS. 23-24 are flowcharts for operation of the UV sterilization device of FIGS. 6-7.

FIG. 25 is a schematic diagram of a UV sterilization device, according to the present disclosure.

FIG. 26 is a schematic diagram of a UV sterilization device, according to the present disclosure.

FIG. 27 is a diagram showing UV LED aging in an embodiment of the UV sterilization device, according to the present disclosure.

FIGS. 28A-28M are images of circuit board layers for the backplane CBA in the UV sterilization device of FIGS. 6-7.

FIGS. 29A-29I are images of circuit board layers for the display in the UV sterilization device of FIGS. 6-7.

FIGS. 30A-30B are images of circuit board layers for the UV CBAs in the UV sterilization device of FIGS. 6-7.

FIGS. 31-33F are circuit diagrams for the controller in an embodiment of the UV sterilization device, according to the present disclosure.

FIGS. 34A-34C are a circuit diagram for power for the controller in an embodiment of the UV sterilization device, according to the present disclosure.

FIGS. 35A-35C are a circuit diagram for the Ethernet connection in an embodiment of the UV sterilization device, according to the present disclosure.

FIGS. 36A-36B are a circuit diagram for the power supplies in an embodiment of the UV sterilization device, according to the present disclosure.

FIG. 37 is a circuit diagram for the detector circuits in an embodiment of the UV sterilization device, according to the present disclosure.

FIGS. 38A-38C are a circuit diagram for the RFID detection in an embodiment of the UV sterilization device, according to the present disclosure.

FIGS. 39A-39F are a circuit diagram for the UV LED drivers in an embodiment of the UV sterilization device, according to the present disclosure.

FIG. 40 is a circuit diagram for the wireless transceiver in an embodiment of the UV sterilization device, according to the present disclosure.

FIG. 41 is a schematic diagram for the flexible connector for the display in an embodiment of the UV sterilization device, according to the present disclosure.

FIGS. 42A-42C are schematic diagrams for the antenna and coaxial connector in an embodiment of the UV sterilization device, according to the present disclosure.

FIG. 43 is several views of the wireless transceiver in an embodiment of the UV sterilization device, according to the present disclosure.

FIGS. 44A-44B are schematic diagrams for the antenna and coaxial connector in an embodiment of the UV sterilization device, according to the present disclosure.

FIG. 45 is a schematic diagram of another embodiment of the UV sterilization system, according to the present disclosure.

FIGS. 46-47 are images of experimental plaques under testing.

FIGS. 48-49 are scanning electron microscope (SEM) images of experimental plaques under testing.

FIGS. 50-53 are diagrams for performance metrics for the plaques under testing.

### DETAILED DESCRIPTION

The present disclosure will now be described more fully hereinafter with reference to the accompanying drawings, in which several embodiments of the invention are shown. This present disclosure may, however, be embodied in many different forms and should not be construed as limited to the embodiments set forth herein. Rather, these embodiments are provided so that this disclosure will be thorough and complete, and will fully convey the scope of the present disclosure to those skilled in the art. Like numbers refer to like elements throughout, and base 100 reference numerals are used to indicate similar elements in alternative embodiments.

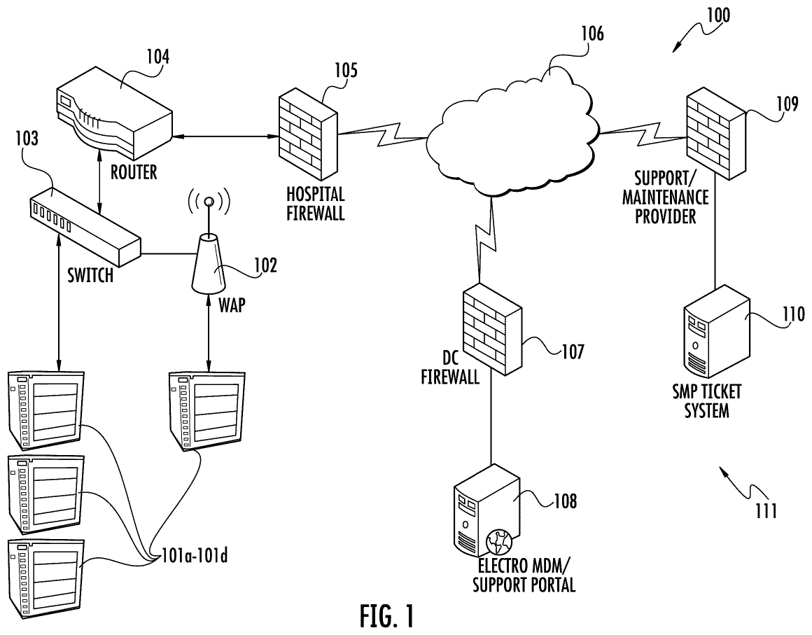

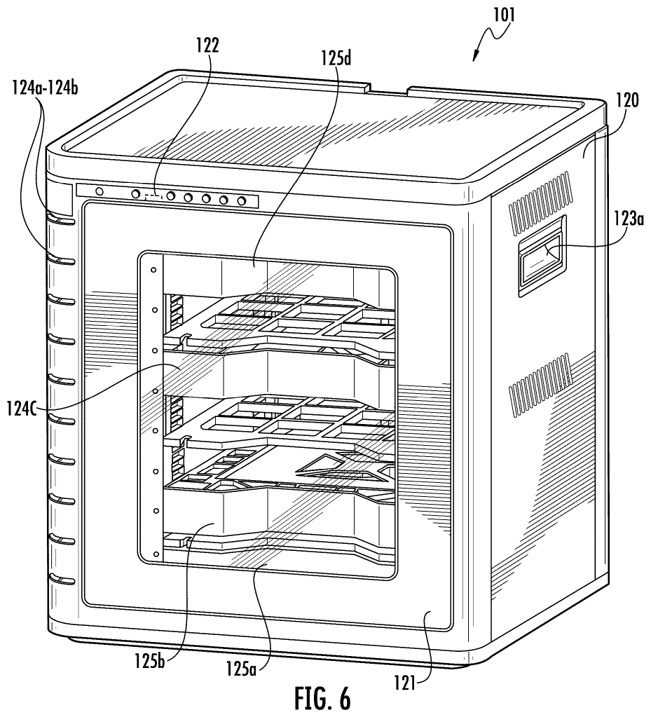

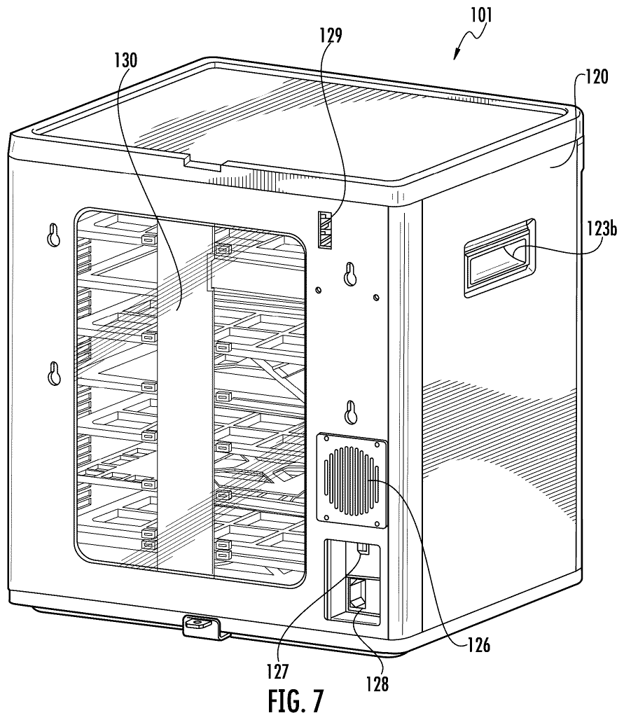

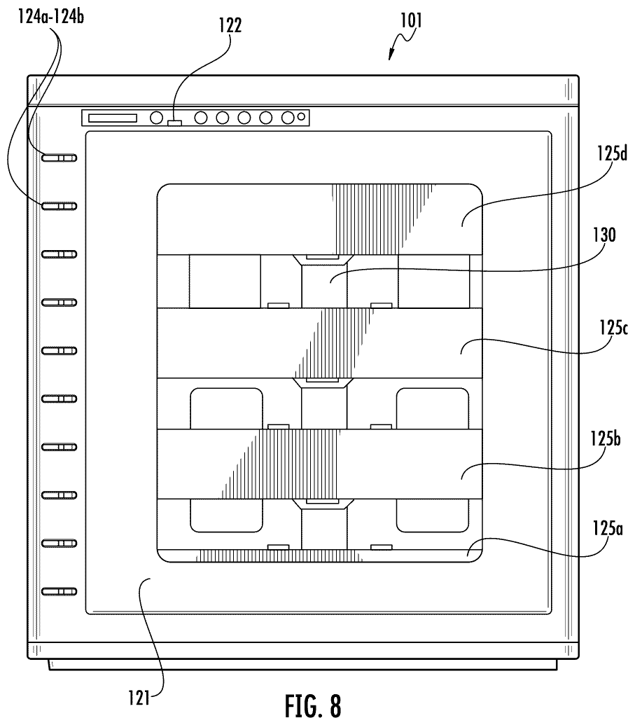

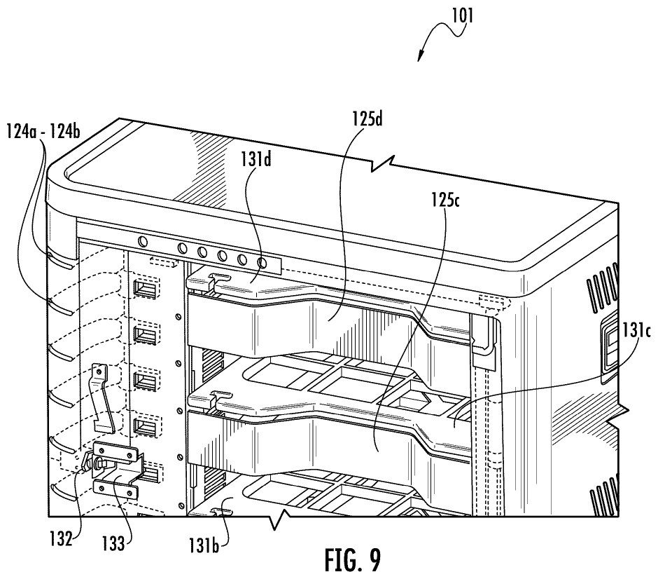

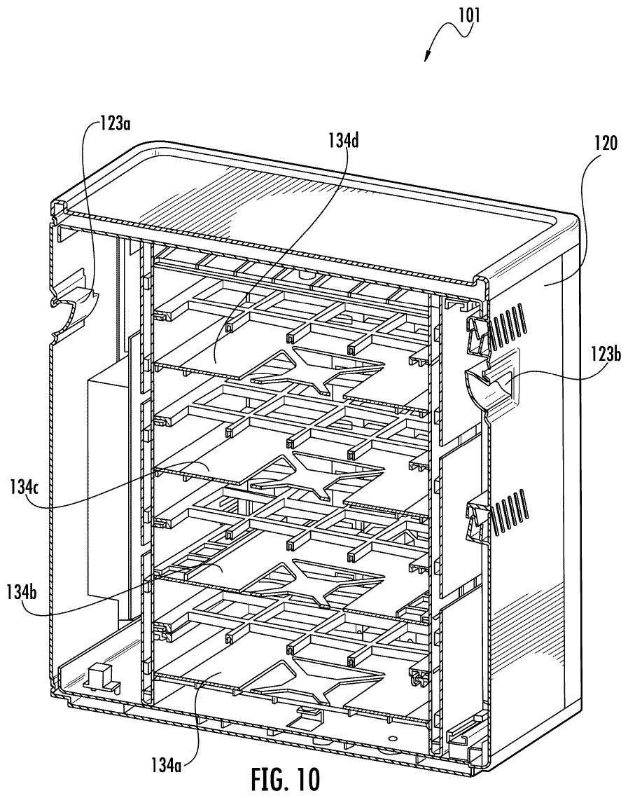

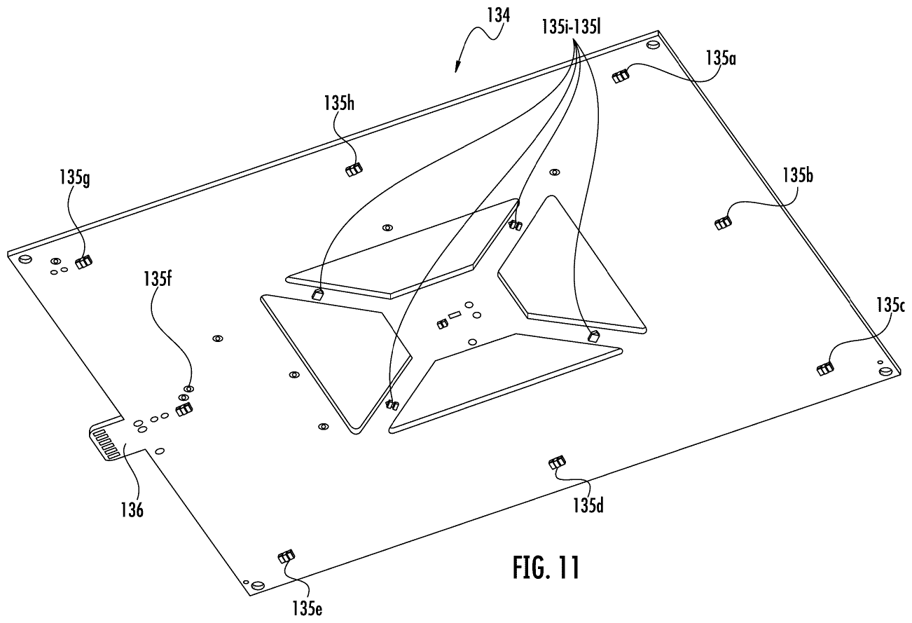

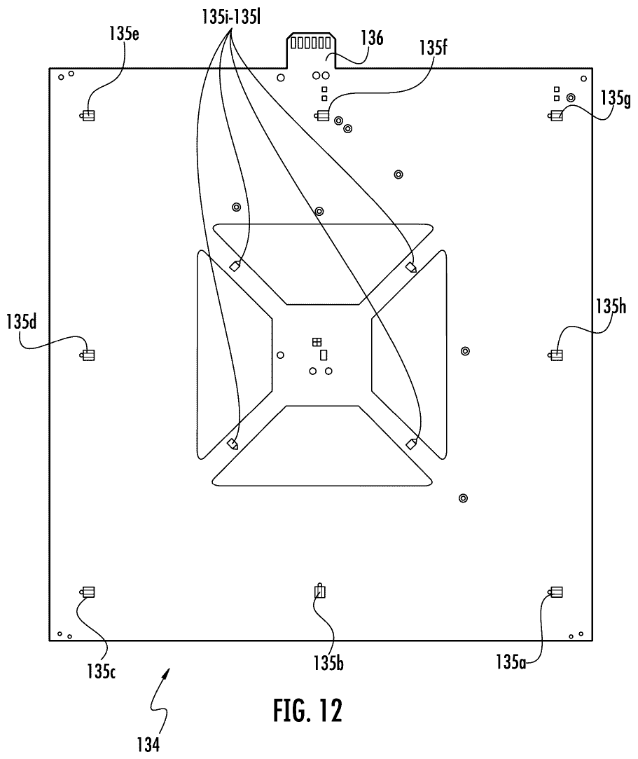

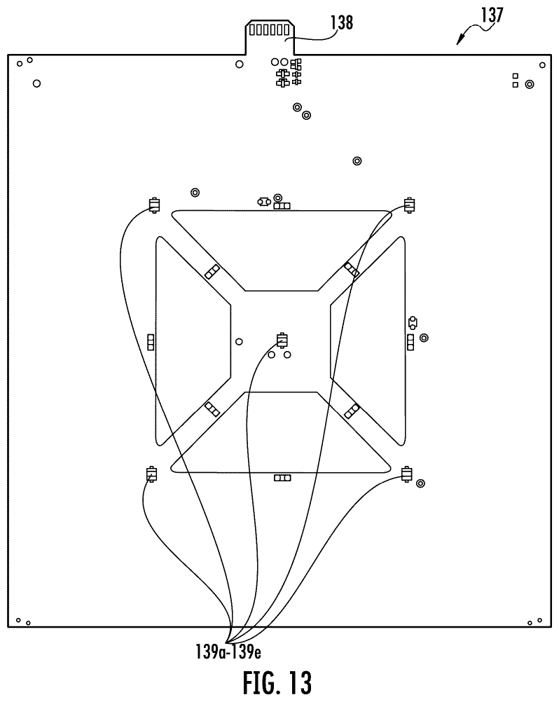

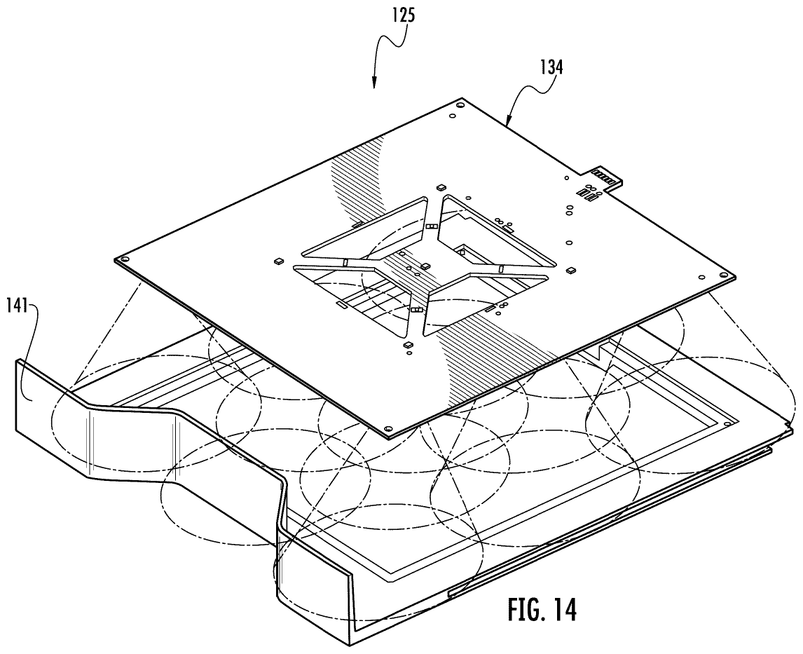

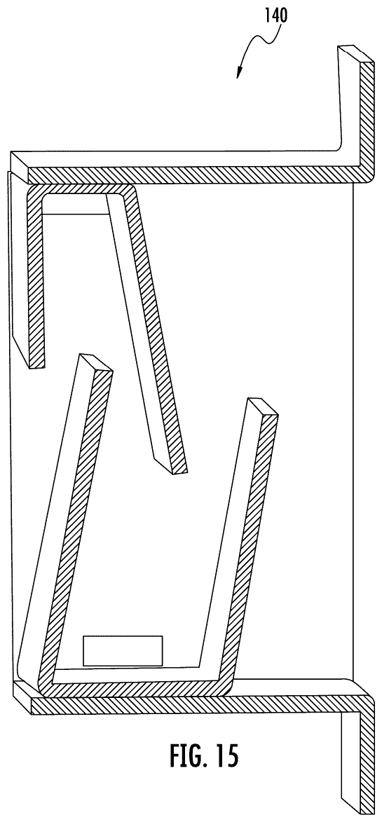

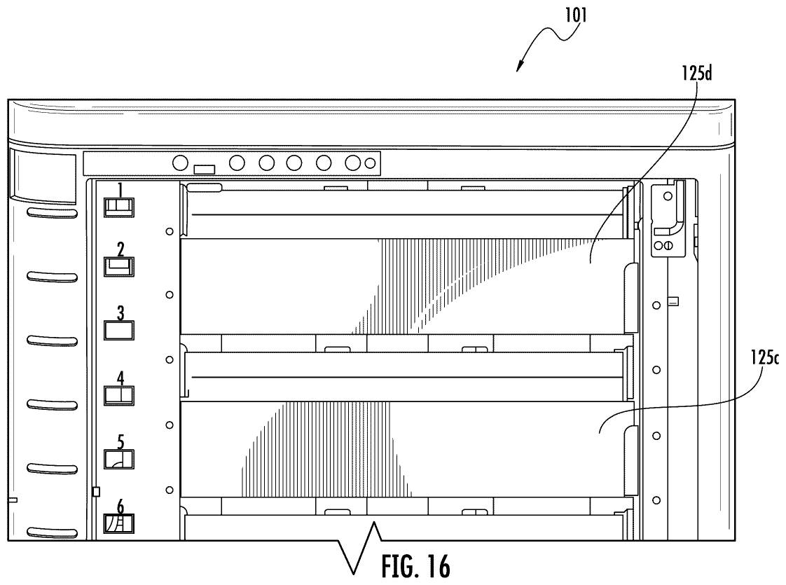

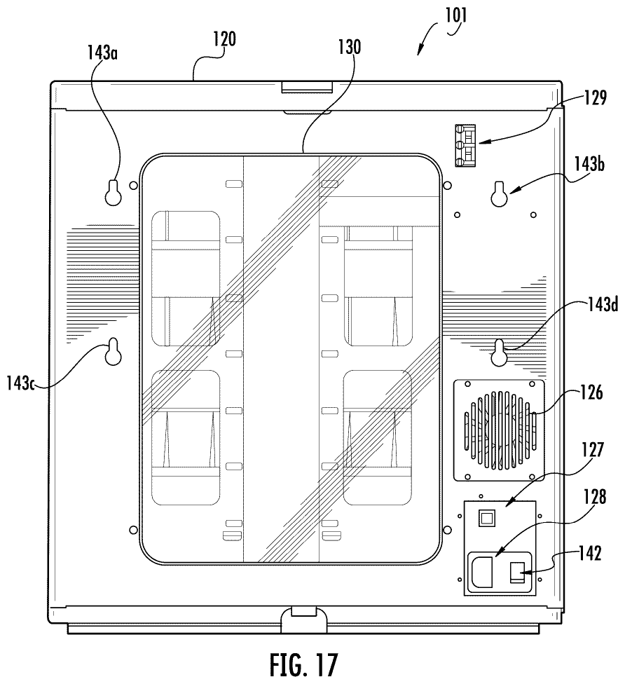

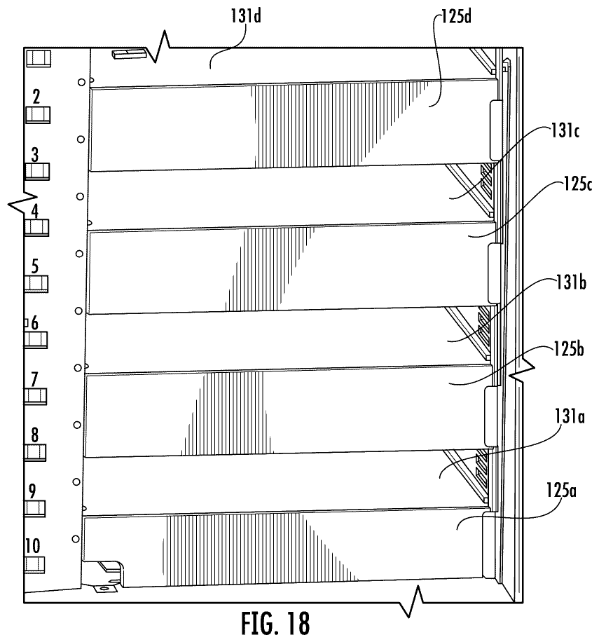

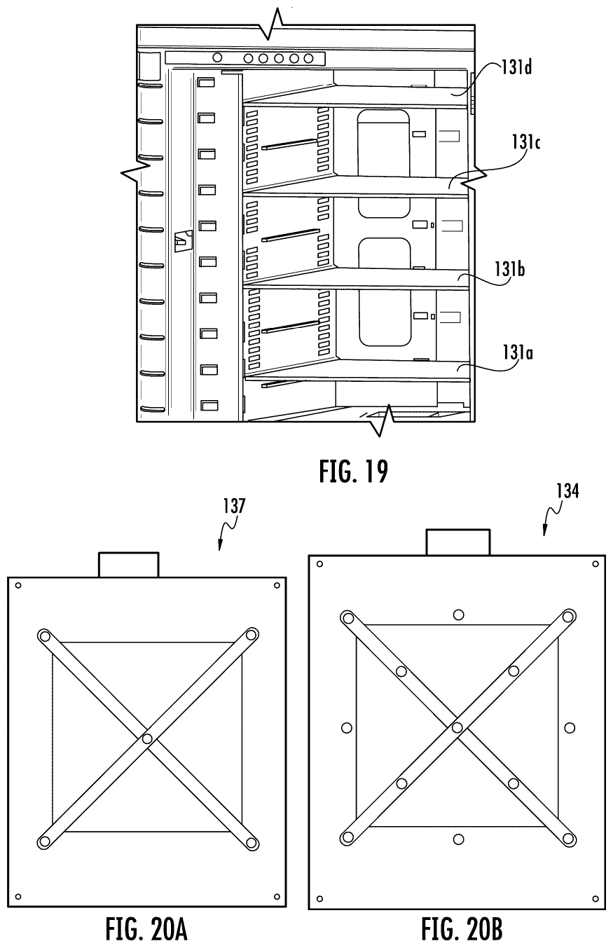

Referring initially to FIGS. 1 and 6-20B, a UV sterilization system 100 according to the present disclosure is now described. The UV sterilization system 100 illustratively includes a server 111 comprising a processor and memory coupled thereto, and a plurality of UV sterilization devices 101a-101d.

In this illustrated embodiment, the server 111 illustratively includes a support/maintenance provider 109 coupled to the Internet 105, a ticket system 110 coupled to the support/maintenance provider, a support portal 108, a firewall 107 coupled between the Internet and the support portal, a hospital firewall 106 coupled to the Internet, a router 104 coupled to the hospital firewall, a switch 103 coupled to the router, a wireless base station 102 coupled to the switch, and the plurality of UV sterilization devices 101a-101d coupled to one or more of the switch or the wireless base station. In other embodiments, the server 111 may be coupled to a local area network, serving only local clients.

Each UV sterilization device 101a-101d illustratively includes a housing 120 defining a cavity therein, and having a door 121 configured to permit access to the cavity. Each UV sterilization device 101a-101d illustratively includes a plurality of trays 131a-131d carried within the cavity, each tray configured to receive at least one mobile wireless communications device.

Each UV sterilization device 101a-101d illustratively includes a plurality of UV CBAs 125a-125d carried within the cavity respectively adjacent the plurality of trays 131a-131d. Each UV CBA 125a-125d illustratively includes a plurality of LED UV sources 135a-1351, 139a-139e configured to irradiate the at least one mobile wireless communications device with UV radiation. Each of the plurality of trays 131a-131d comprises a plurality of device bays for receiving devices of varying sizes and configurations (e.g. cell phones, tablets, laptops). As will be appreciated, the UV irradiation eliminates microbial contamination on the at least one mobile wireless communications device, i.e. effecting a disinfection or sanitizing process.

Each UV sterilization device 101a-101d illustratively includes a transceiver (e.g. Wifi, Bluetooth, ZigBee, cellular) 153 configured to communicate with the server 111, and a controller 144 configured to selectively power at least one of the plurality of UV CBAs 125a-125d for disinfecting the at least one mobile wireless communications device, and send at least one status message to the server. The server 111 is configured to contact respective users via e-mail or short message service (SMS) messages when needed. Advantageously, the UV sterilization device 101a-101d is more power efficient since unused trays 131a-131d are not irradiated.

Each tray 131a-131d comprises a material transparent to UV radiation. For example, the material comprises quartz. Indeed, since the plurality of LED UV sources 135a-1351 have a narrow emission spectrum, the material need only be transparent to the emission spectrum and not the entire UV band. Helpfully, since there are no UV shadows on the device, this permits for 360 degree irradiation of the at least one mobile wireless communications device.

The server 111 is be configured to provide a web interface portal for accessing respective status information for the plurality of UV sterilization devices 101a-101d. The at least one mobile wireless communications device includes an RFID tag. The UV sterilization device 101a-101d illustratively includes an RF transmitter (e.g. an ultra high frequency (UHF) transmitter) 115 coupled to the controller 144 and configured to energize the RFID tag. The controller 144 is configured to identify the at least one mobile wireless communications device based upon an RF signal from the RFID tag. The controller 144 is configured to selectively power the at least one of the plurality of UV CBAs 125a-125d when the at least one mobile wireless communications device is detected on a respective tray. In these embodiments, each mobile wireless communications device would have a passive RFID tag, for example, an adhesive backed RFID tag.

Advantageously, the RF transmitter 115 is configured to operate in a closed loop system, and segregates device bays in each tray 131a-13d into an RF zone to solve for device orientation and placement. In fact, some embodiments may include a plurality of RF transmitters, which are activated in sequence based upon the detected placement and number of mobile wireless communications devices within the cavity. In these embodiments, the plurality of RF transmitters may operate with circular polarization.

Also, this identification feature permits the UV sterilization device 101a-101d to maintain an accurate real-time inventory of the mobile wireless communications devices within the plurality of trays 131a-13d. This may aid in providing for reduction of device theft and loss. Further, the disinfection status and frequency of each mobile wireless communications device is monitored and reported back to the server 111.

The UV sterilization device 101a-101d may further comprise a keypad carried on an external surface of the housing 120 and coupled to the controller 144. The controller 144 is configured to unlock the door 121 based upon a code received from the keypad. Since each user has a unique code to open the door 121, the UV sterilization device 101a-101d can keep track of which user opened the door and verify the proper device was removed. Indeed, if the wrong device is removed, the UV sterilization device 101a-101d may cause the server 111 to send a notification to the user.

In other embodiments, the UV sterilization device 101a-101d may further comprise a near field communications (NFC) device configured to communicate with the mobile wireless communications device. Helpfully, the NFC device would be accessible from the exterior of the UV sterilization device 101a-101d and permit the user to tap the mobile wireless communications device for access. In other words, the UV sterilization device 101a-101d may identify the mobile wireless device and open the door 121 when the NFC device detects an authorized device.

The UV sterilization device 101a-101d may further comprise a charging port (e.g. universal serial bus) carried within the cavity and configured to charge the at least one mobile wireless communications device. Also, in the cavity, the housing 120 comprises a plurality of cable management devices for reducing potential UV shadow of charging cables.

Helpfully, the UV sterilization device 101a-101d use of LED UV light sources creates less waste infrared (IR), i.e. heat, energy, keeping the at least one mobile wireless communications device at a lower temperature during the disinfection process. In prior approaches that used incandescent UV sources, the at least one mobile wireless communications device would be heated with waste IR energy, making simultaneous charging undesirable or impossible.

The housing 120 comprises opposing front and back sides, the front side defining the door 121. The UV sterilization device 101a-101d may further comprise a backplane CBA 130 carried by the back side and configured to receive and power the plurality of UV CBAs 125a-125d. In fact, the plurality of UV CBAs 125a-125d are readily removable and coupled to the backplane CBA via a slot connection. In the illustrated embodiment, each UV CBAs 125a-125d illustratively includes a circuit board 134, 137 comprising a connector tab 136, 138 to be received by respective slots in the backplane CBA 130, and a carrying housing 141 coupled to the circuit board. The carrying housing 141 illustratively includes side edges to be received by the slots within the sides of the cavity of the housing 120.

The UV sterilization device 101a-101d illustratively includes a positive air pressure source 126 (e.g. an electrical fan) carried by the housing and configured to create positive air pressure in the cavity when the door 121 is open. Helpfully, this causes an outflow of air when the door 121 is open and prevents particulate contaminants from entering the cavity, which can cause UV micro-shadowing during the disinfection process. In some embodiments, the positive air pressure source 126 illustratively includes an air filter (e.g. HEPA filter) to filter incoming air, preventing unintended ingestion of particulate matter from the positive air pressure source. In some embodiments, the housing 120 comprises a plurality of adjustable feet on a bottom exterior surface.

The UV sterilization device 101a-101d may further comprise a sensor (e.g. Hall effect sensor) coupled to the controller 144 and configured to detect a state of the door 121. Accordingly, when the door 121 is opened, the controller sends a status update to the server 111. The UV sterilization device 101a-101d illustratively includes a latch 133, and an electromechanical solenoid 132 cooperating therewith and coupled to the controller 144. The electromechanical solenoid 132 is configured secured the door 121. In embodiments where the UV sterilization device 101a-101d has a keypad for coded entry, the controller 144 would include a user identifier in the status message, and would activate the solenoid 132 to open the door 121. In these embodiments, the door 121 is biased to open outward with a spring, for example. In some embodiments, the controller 144 may activate a sound indicator to alert a user to when the door 121 is open for a period greater than a set timeout period. In some embodiments, the door 121 may be totally automated and motorized to open and close.

The UV sterilization device 101a-101d may further comprise a data communications bus coupled to the controller 144, and a plurality of wired data ports (e.g. Ethernet) 129 coupled to the data communications bus. The UV sterilization device 101a-101d illustratively includes a display 122 coupled to the controller 144 and configured to present operational indicators to the user. In some embodiments, the keypad may be integrated with the display 122. The UV sterilization device 101a-101d illustratively includes a pair of handles 123a-123b on sides of the housing 120 and configured to permit easy handling. The UV sterilization device 101a-101d illustratively includes a plurality of indicators 124a-124b for indicating a charging status for the at least one mobile wireless communications device. As shown, the door 121 illustratively includes a plurality of light conduits coupled to internal indicator LEDs.

The UV sterilization device 101a-101d illustratively includes a wired port 129 (e.g. universal serial bus (USB) B type) configured to permit access to the controller 144 for firmware updates and maintenance, a power connector 128 configured to receive a power cord, and a power switch 127 configured to power toggle the device. The UV sterilization device 101a-101d illustratively includes a light louver 140 configured to permit air to flow out of cavity, but prevent UV radiation from leaking out. The UV sterilization device 101a-101d illustratively includes a plurality of wall mount openings 143a-143d configured to anchor the housing 120 to a wall.

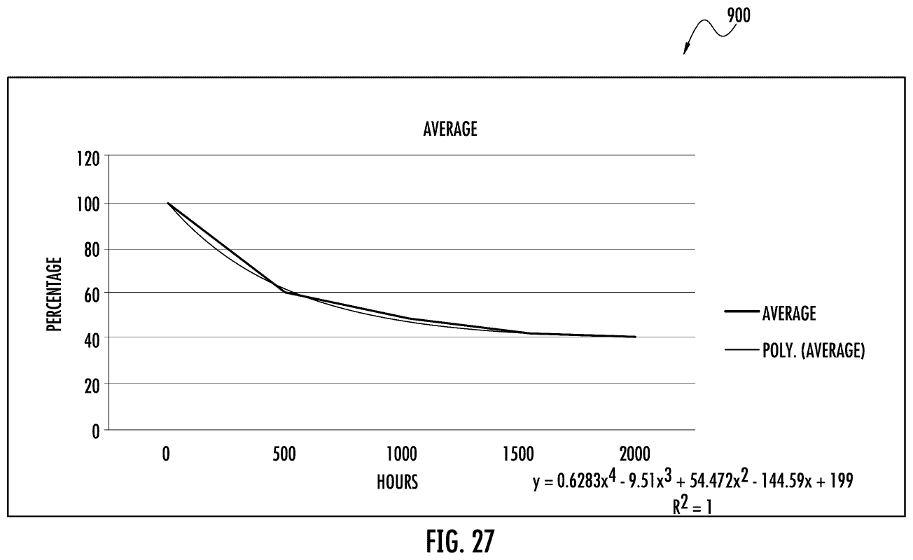

The UV sterilization device 101a-101d illustratively includes an LED driver circuit configured to drive the UV LEDS with a stepped waveform, which provides for a constant operation current and a narrow emission spectrum. This also provides for a long lifespan for the UV LEDs, on the order of 5,000 hours. Moreover, the controller 144 is configured to monitor the wear on the UV LEDs and alert the server 11 when replacement is needed. In particular, the wear on the UV LEDs is based upon a known degradation curve, as show in diagram 900 (FIG. 27).

Another aspect is directed to a method for operating a UV sterilization device 101a-101d including a housing 120 defining a cavity therein, and having a door 121 configured to permit access to the cavity, and a plurality of trays 131a-131d carried within the cavity, each tray configured to receive at least one mobile wireless communications device. The UV sterilization device 101a-101d may include a plurality of UV CBAs 125a-125d carried within the cavity respectively adjacent the plurality of trays, each UV CBA comprising a plurality of LED UV sources 135a-1351 configured to irradiate the at least one mobile wireless communications device with UV radiation. The UV sterilization device 101a-101d may include a controller 144, and the method may include operating the controller to selectively power at least one of the plurality of UV CBAs 125a-125d for disinfecting the at least one mobile wireless communications device.

Another aspect is directed to a method for operating a UV sterilization system 100 comprising a server 111 comprising a processor and memory coupled thereto, and a plurality of UV sterilization devices 101a-101d. Each UV sterilization device 101a-101d may include a housing 120 defining a cavity therein, and having a door 121 configured to permit access to the cavity, a plurality of trays 131a-131d carried within the cavity, each tray configured to receive at least one mobile wireless communications device, and a plurality of UV CBAs 125a-125d carried within the cavity respectively adjacent the plurality of trays. Each UV CBA 125a-125d may comprise a plurality of LED UV sources 135a-1351 configured to irradiate the at least one mobile wireless communications device with UV radiation. Each UV sterilization device 101a-101d may comprise a transceiver 153 configured to communicate with the server 111. The method may include operating a controller 144 to selectively power at least one of the plurality of UV CBAs 125a-125d for disinfecting the at least one mobile wireless communications device, and send at least one status message to the server 111. The method may include operating the server 111 to provide a web interface portal for accessing respective status information for the plurality of UV sterilization devices 101a-101d.

Advantageously, controller 144 is configured with a custom firmware. This is advantageous in the healthcare facility application for the following reasons. Since prior approaches may leverage existing operating systems (OSs) to cut costs and provide built-in functionality, they are rarely updated once put into use as medical devices, which can lead to critical unpatched vulnerabilities. This may cause security risks to local networks in healthcare facilities.

The custom firmware of the controller 144 addresses this issue, yet includes a Transmission Control Protocol/Internet Protocol (TCP/IP) stack to communicate on typical networks. In particular, the controller is configured to perform one-time handshakes with the server 111, the switch 103, and/or the wireless base station 102. The controller is configured to provide for media access control address (MAC address) authentication, so that the handshake occurs only with proper devices.

Also, the UV sterilization device 101a-101d illustratively includes a plurality of image sensors configured to detect a number and location of mobile wireless communications devices placed in the cavity. Advantageously, the controller 144 then activates needed antennas of the RF transmitter 115 to activate the respective RFID tag on the device. For example, the plurality of image sensors may each comprise an RGB color sensor, or a proximity sensor.

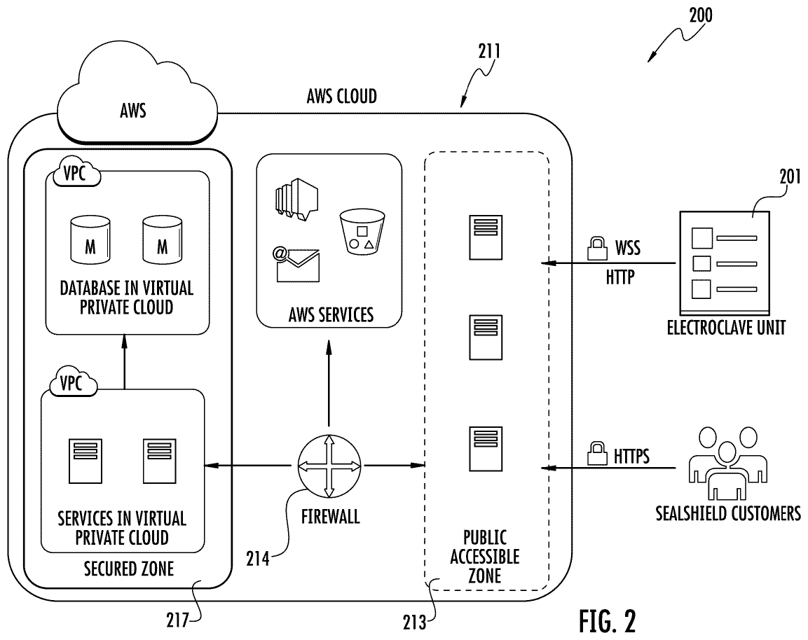

Referring now additionally to FIG. 2, another embodiment of the UV sterilization system 100 is now described. In this embodiment of the UV sterilization system 200, those elements already discussed above with respect to FIG. 1 are incremented by 100 and most require no further discussion herein. This embodiment differs from the previous embodiment in that this UV sterilization system 200 illustratively includes the server 211 as a cloud based service provided by Amazon Web Services. The server 211 illustratively includes a public accessible zone 212 providing the web interface, a secured zone 212, and a firewall 214.

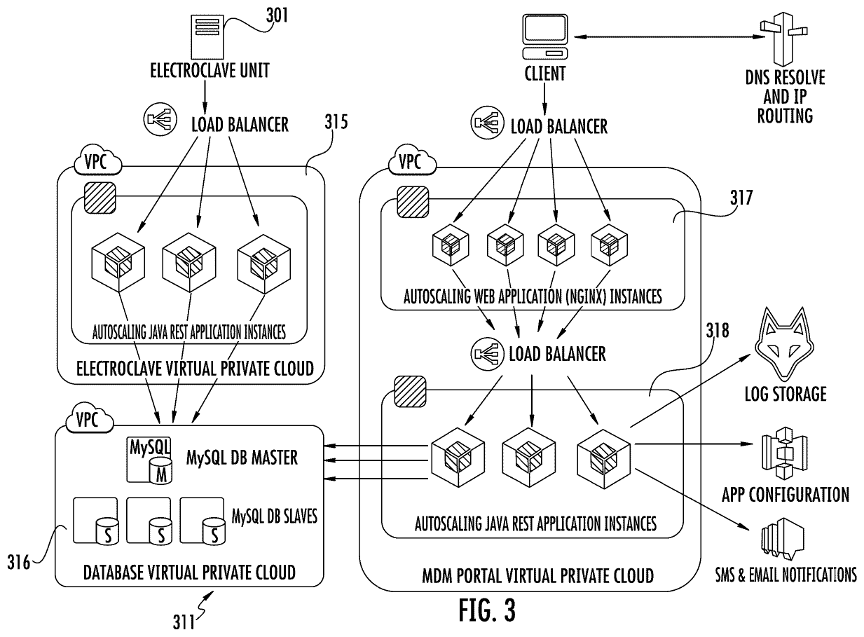

Referring now additionally to FIG. 3, another embodiment of the UV sterilization system 100 is now described. In this embodiment of the UV sterilization system 300, those elements already discussed above with respect to FIG. 1 are incremented by 200 and most require no further discussion herein. This embodiment differs from the previous embodiment in that this UV sterilization system 300 illustratively includes the server 311 as a virtual private cloud implementation. The server 311 illustratively includes a plurality of modules 315-318.

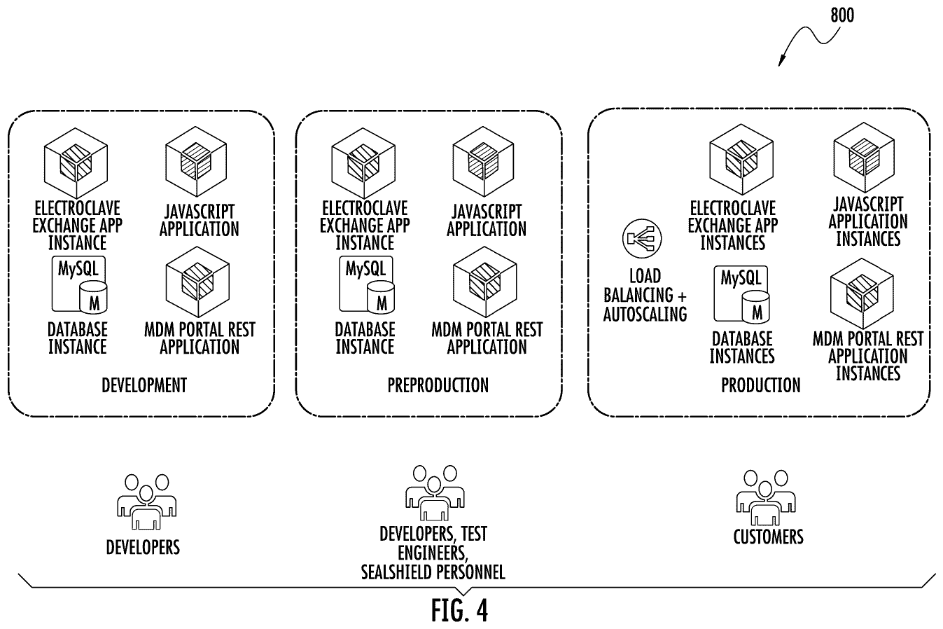

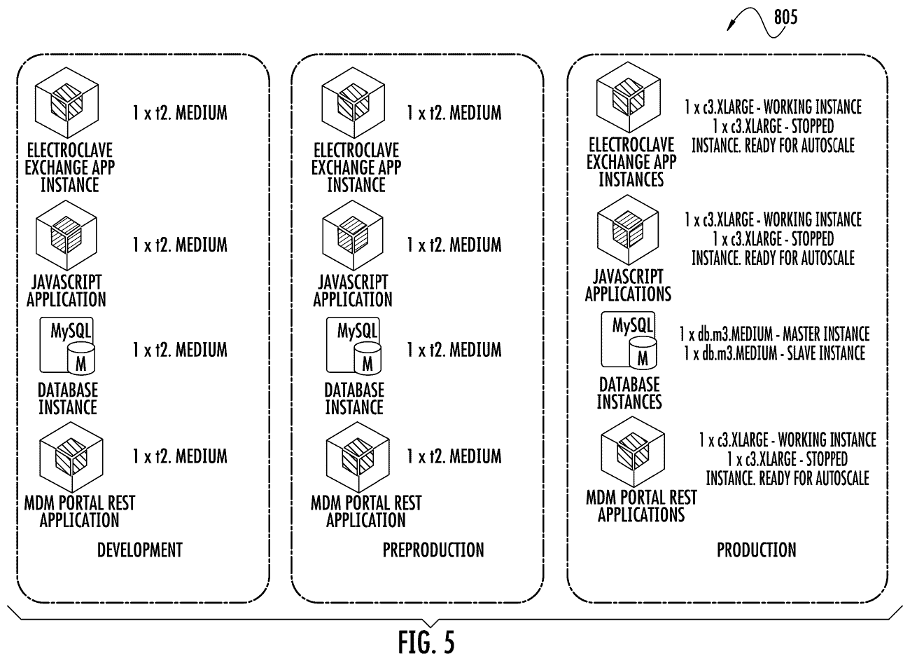

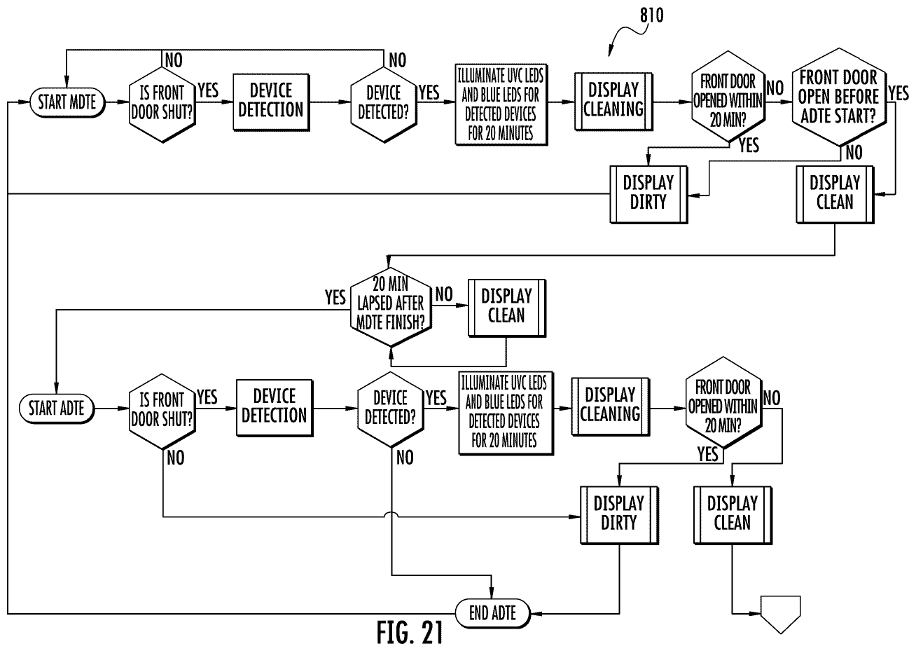

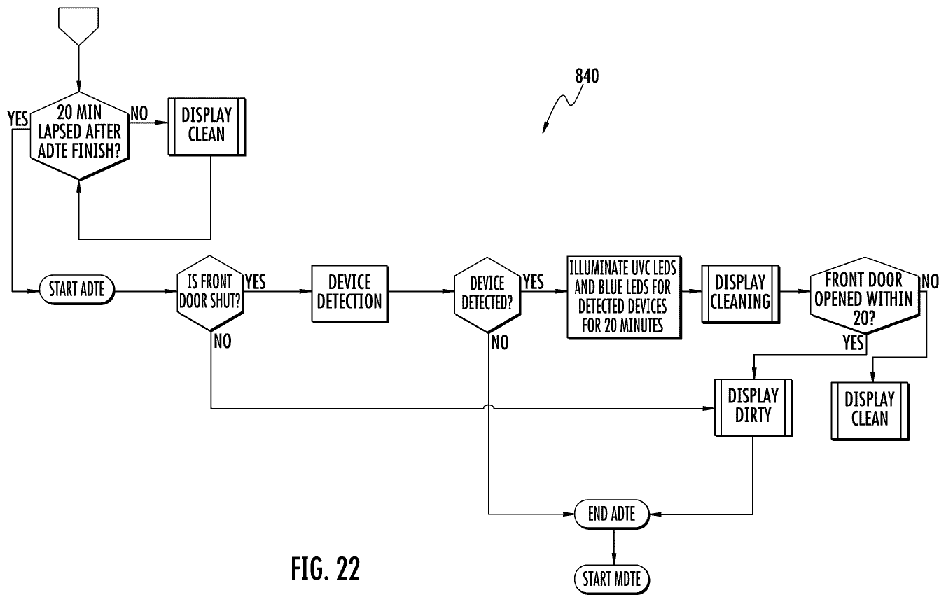

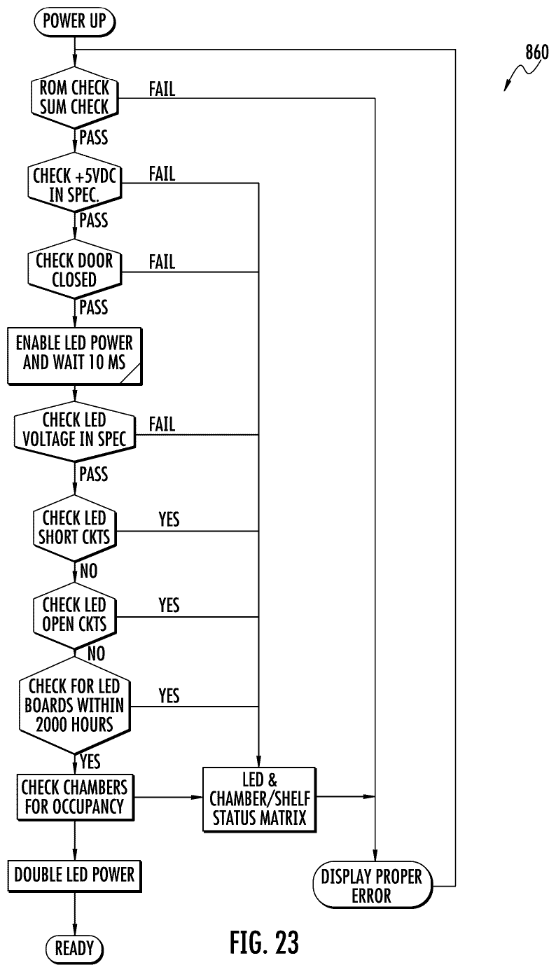

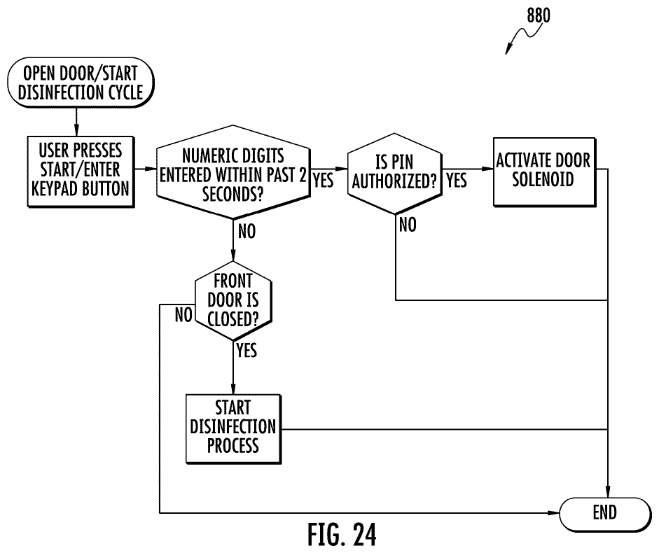

Referring now to FIGS. 4-5, 21-24, diagrams 800, 805 illustrate structure of example embodiments of the UV sterilization system 100. Flowcharts 810, 840, 860, 880 show the logic of the controller 144 of the operation of the UV sterilization device 101a-101d.

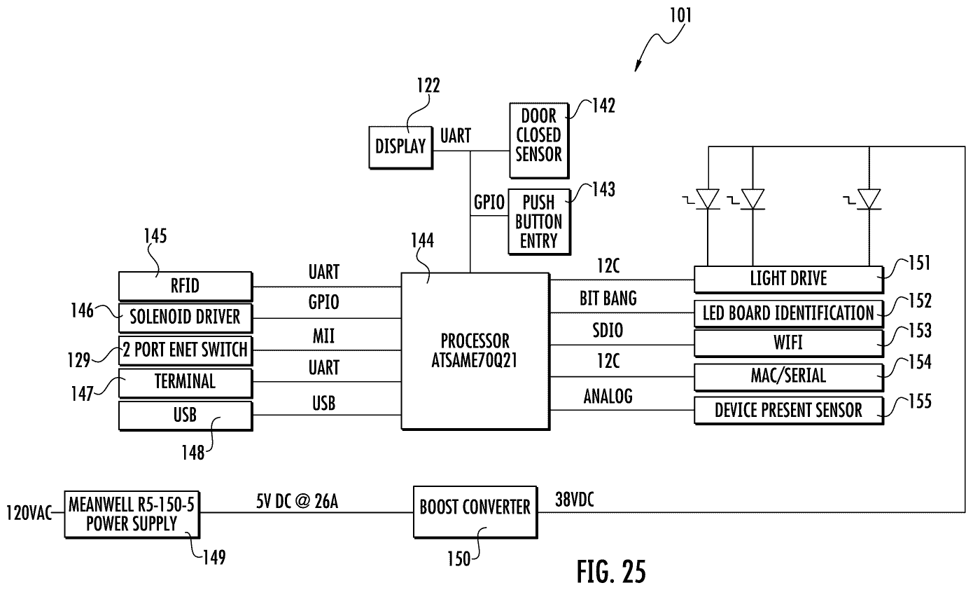

Referring now to FIG. 25, the UV sterilization device 101 illustratively includes the RF transmitter 115 coupled to the controller 144, the solenoid driver circuit 146 coupled to the controller, the wired port 129 coupled to the controller, a terminal port 147 also coupled to the controller, a display 122 coupled to the controller, a door sensor 142 coupled to the controller, and a push button entry 143 coupled to the controller. The UV sterilization device 101 illustratively includes a light driver circuit 151 coupled to the controller 144, a LED board identification circuit 152 coupled to the controller, a wireless transceiver 153 coupled to the controller, a MAC/serial circuit 154 coupled to the controller, and a device present sensor (e.g. image sensor) coupled to the controller. The UV sterilization device 101 illustratively includes a power supply 149, and a boost converter circuit 150 coupled between the power supply and the light driver circuit 151.

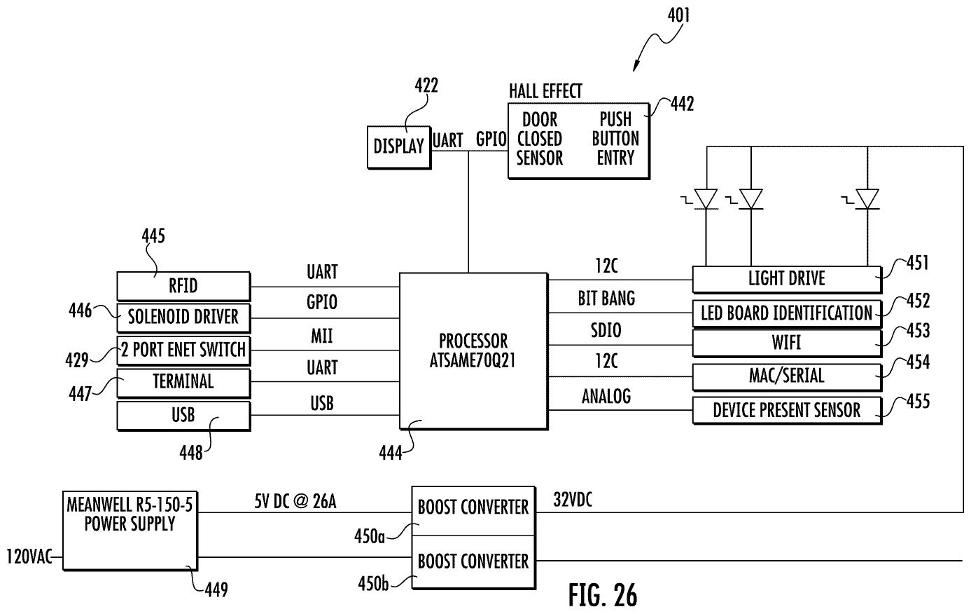

Referring now additionally to FIG. 26, another embodiment of the UV sterilization device 101 is now described. In this embodiment of the UV sterilization device 401, those elements already discussed above with respect to FIG. 25 are incremented by 300 and most require no further discussion herein. This embodiment differs from the previous embodiment in that this UV sterilization device 401 illustratively includes first and second boost converters 450a-450b.

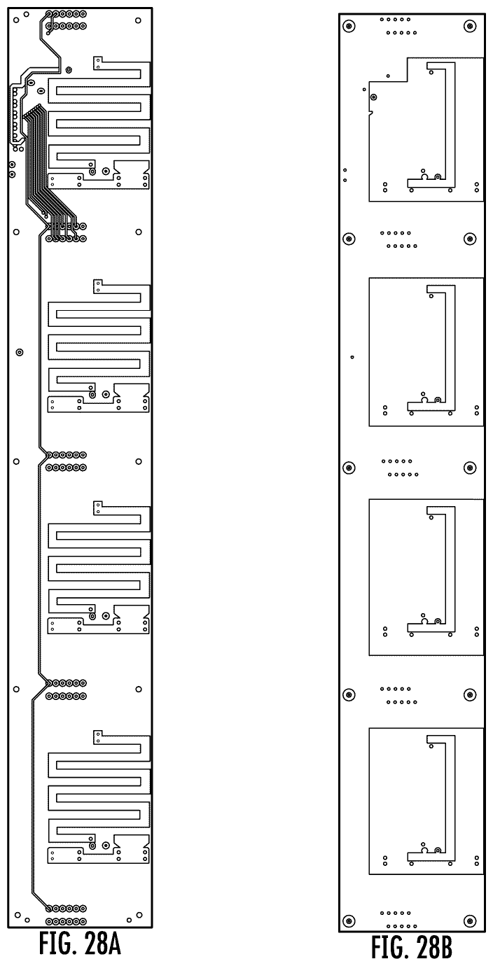

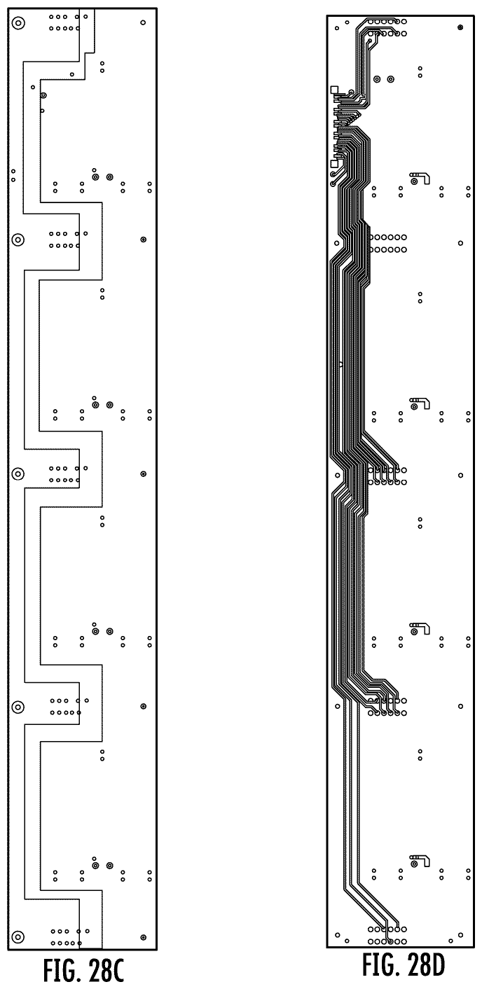

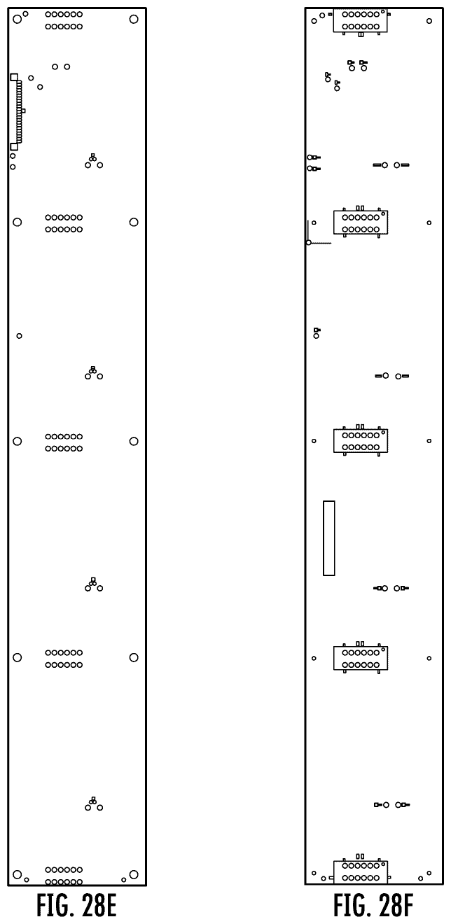

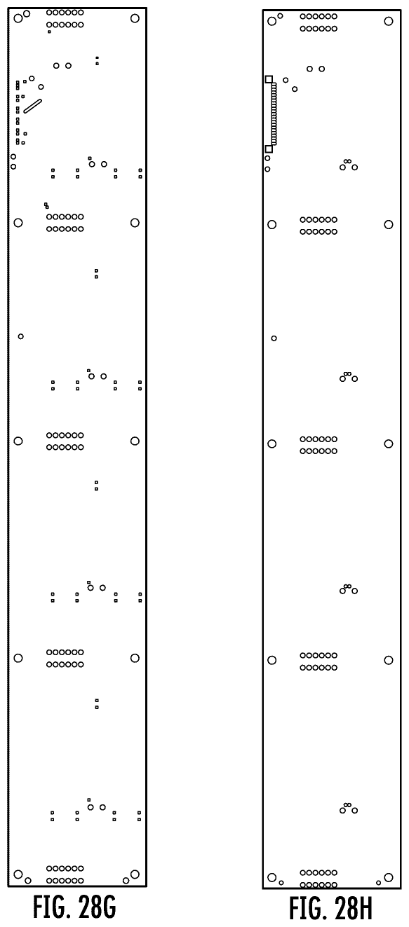

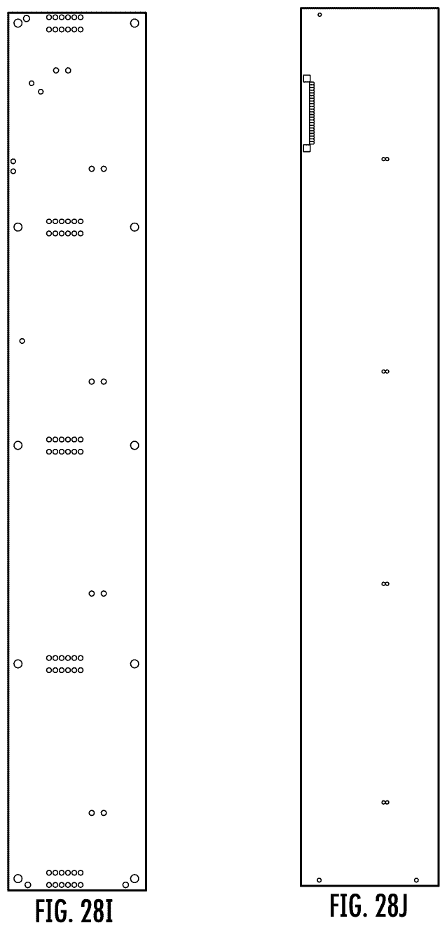

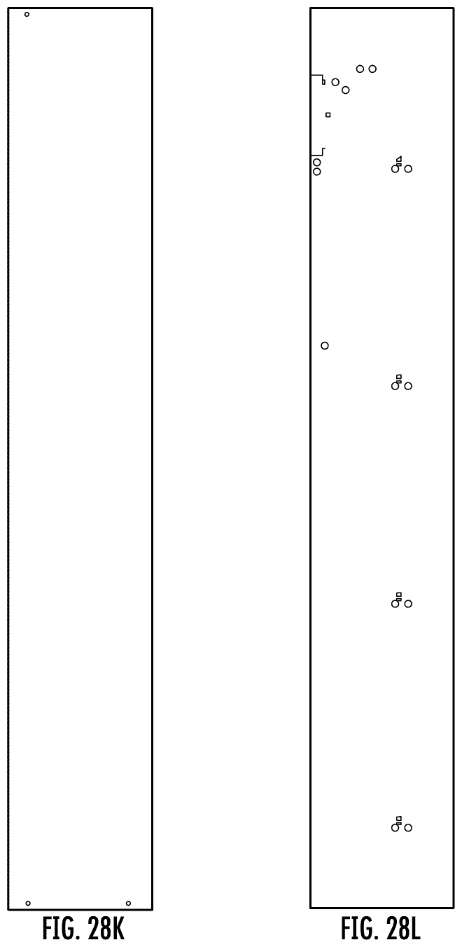

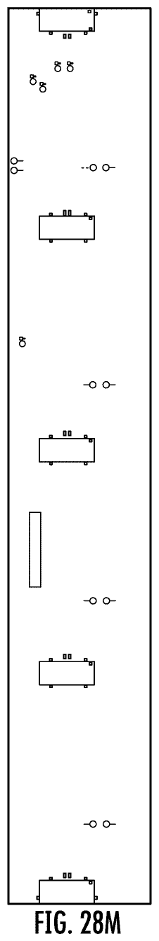

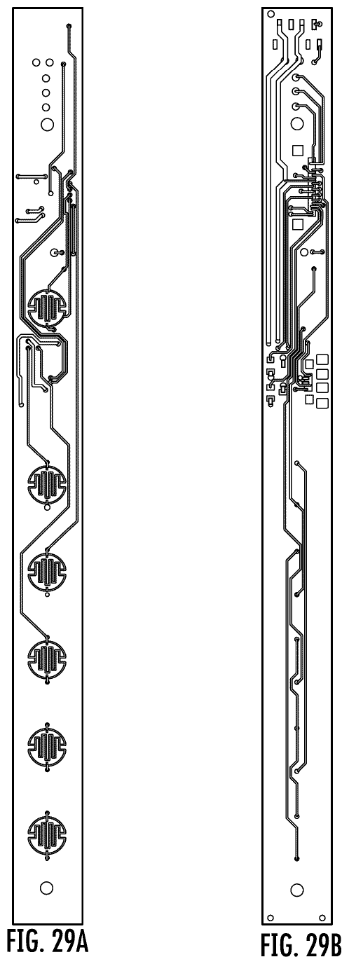

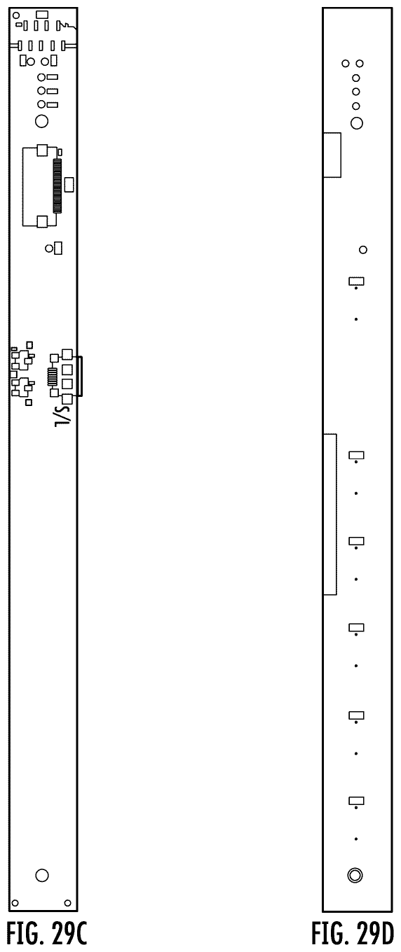

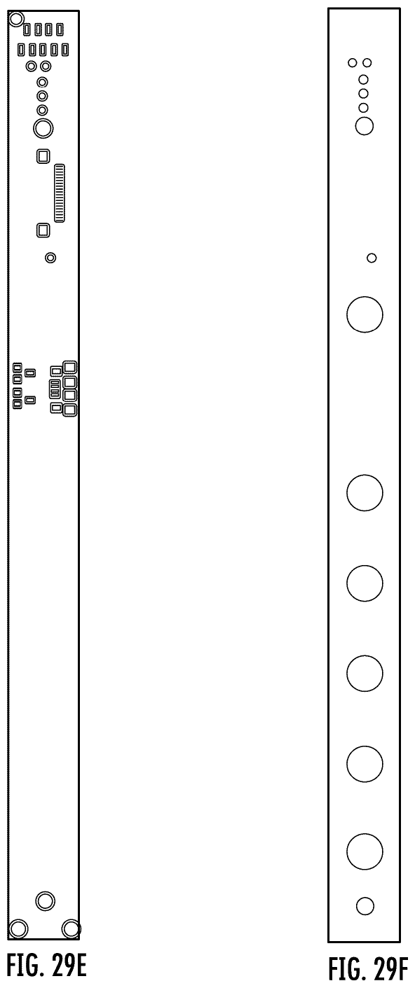

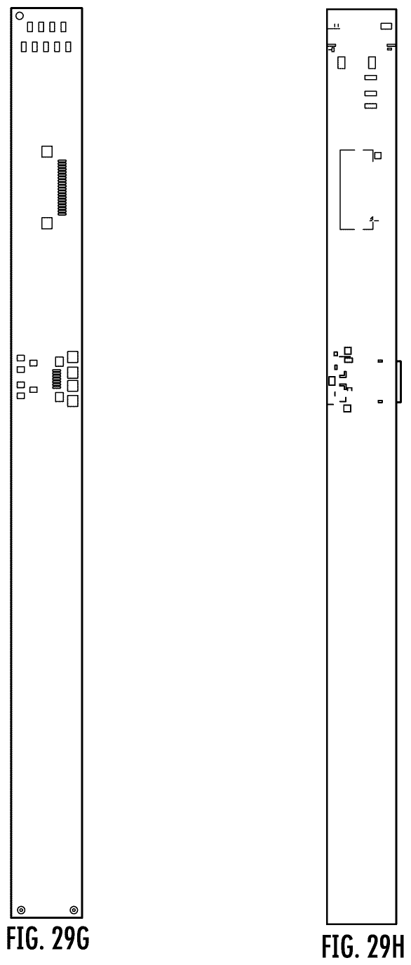

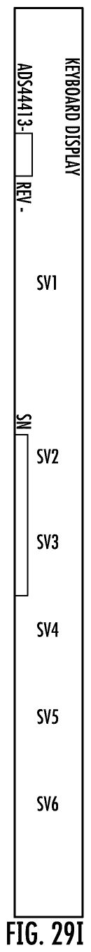

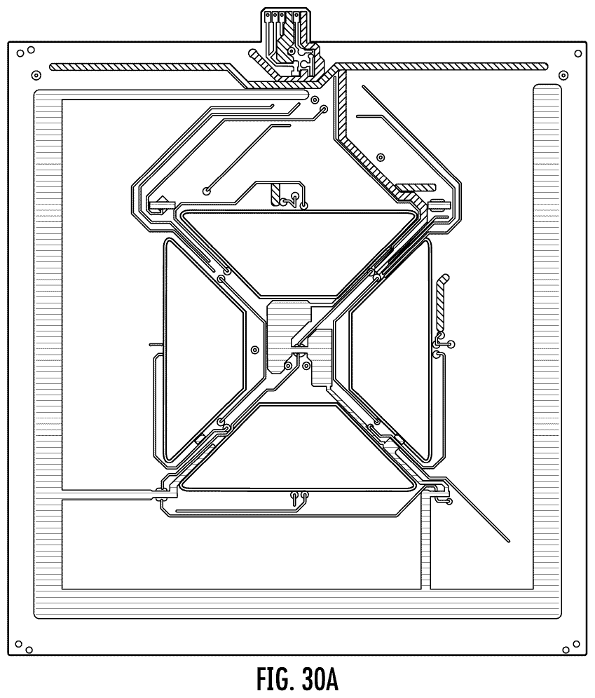

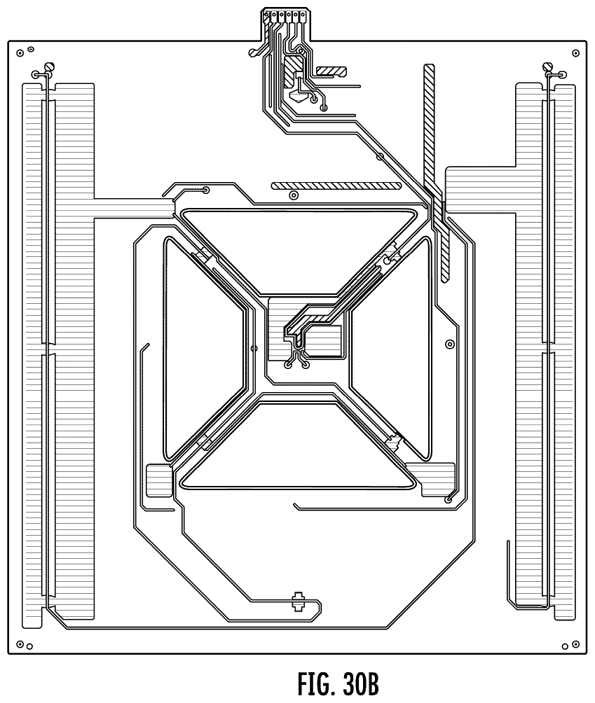

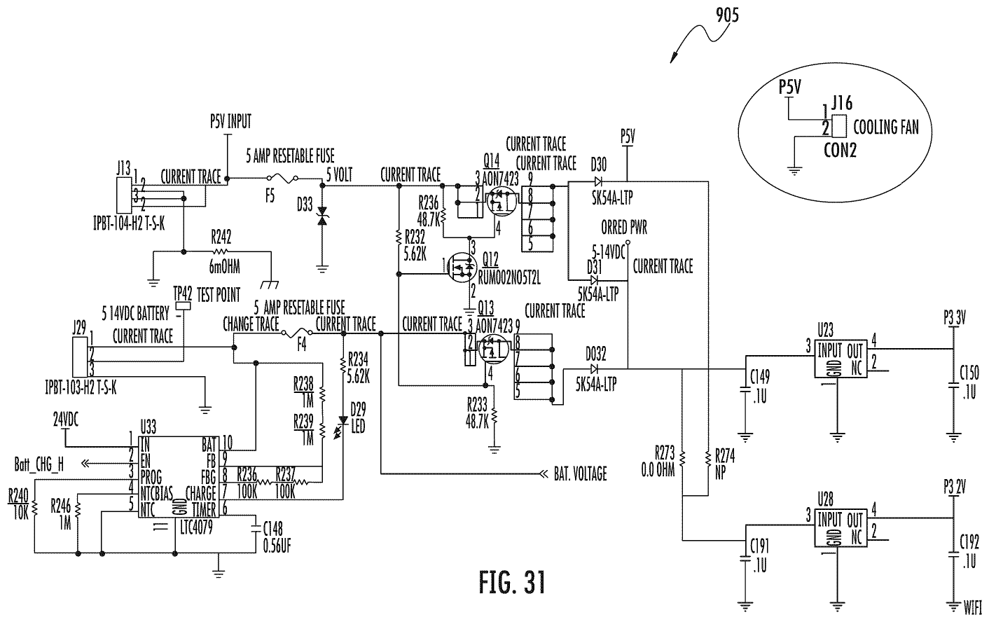

Referring to FIGS. 28A-28M, patterns for circuit board layers for the backplane CBA 130 in the UV sterilization device 101a-101d are shown. Referring to FIGS. 29A-29I, patterns for circuit board layers for the display in the UV sterilization device 101a-101d are shown. Referring to FIGS. 30A-30B, patterns for circuit board layers for the UV CBAs 125a-125d in the UV sterilization device 101a-101d are shown.

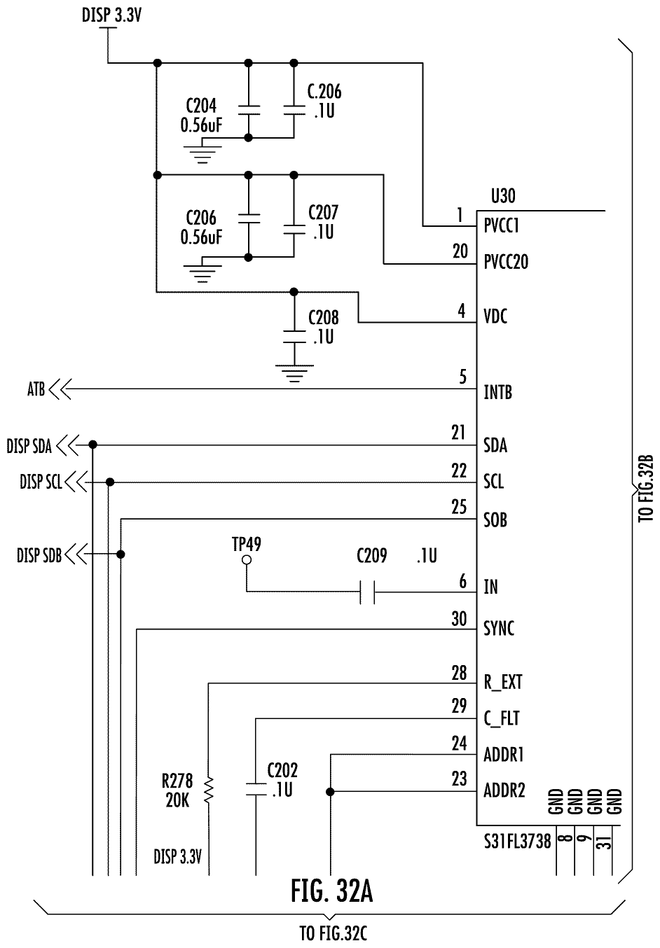

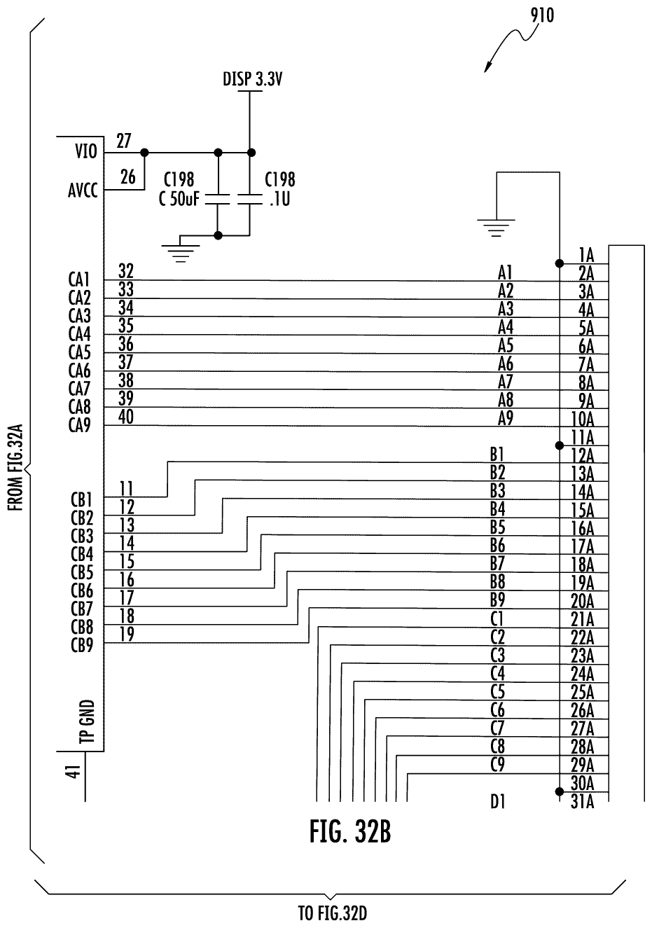

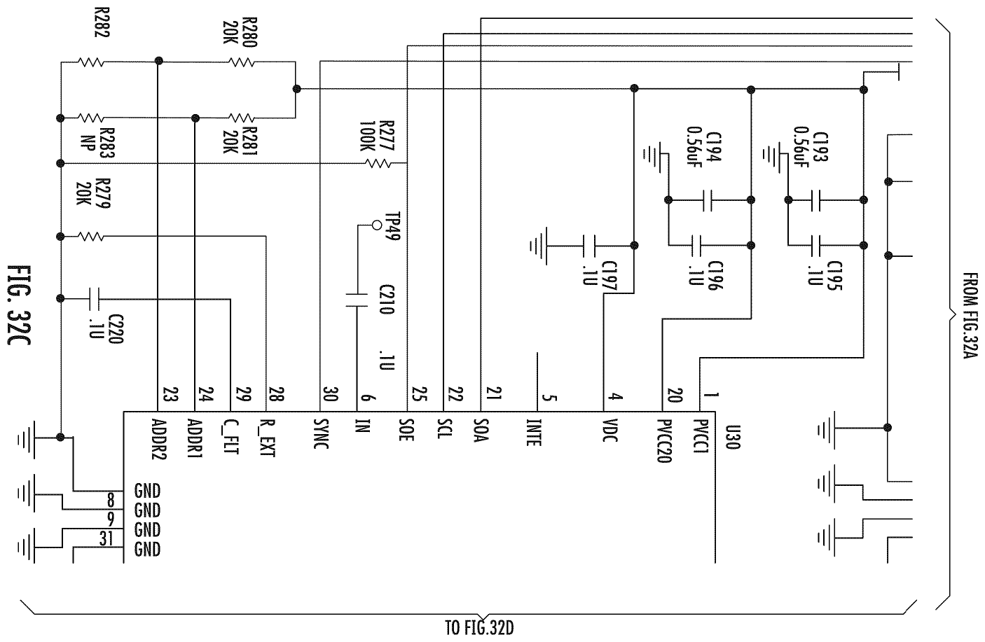

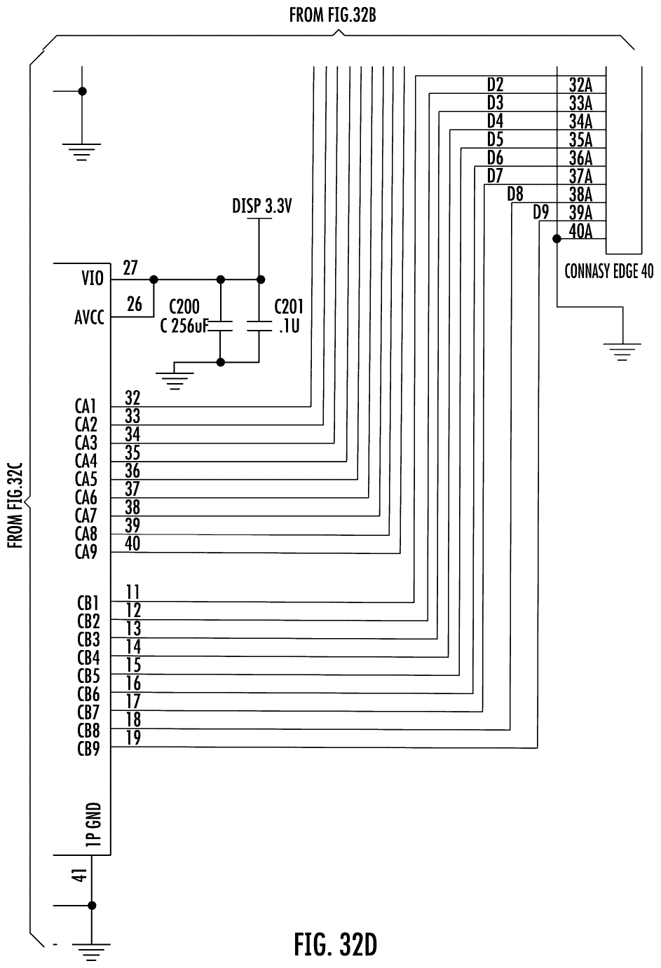

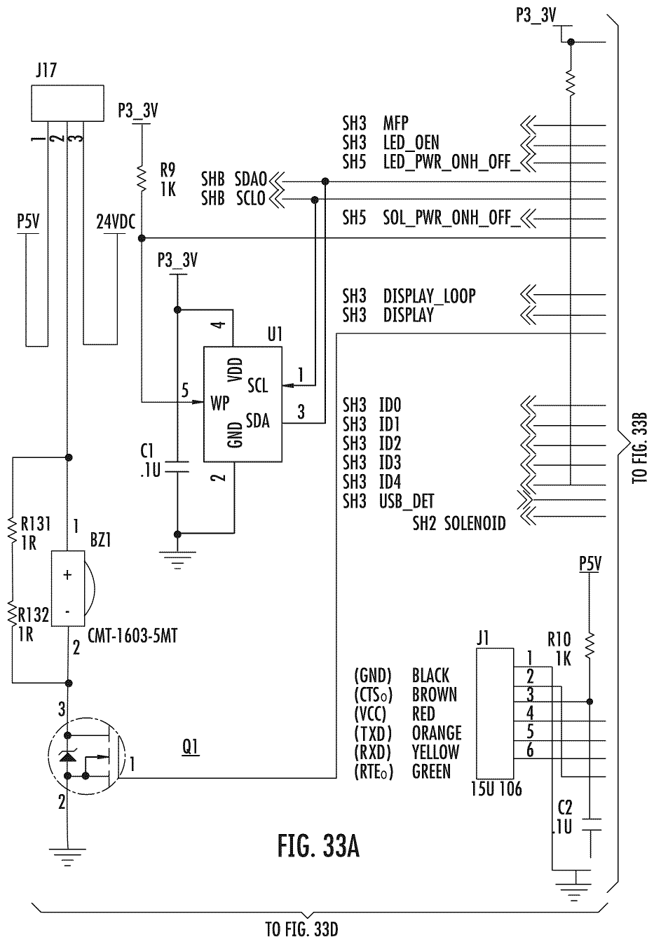

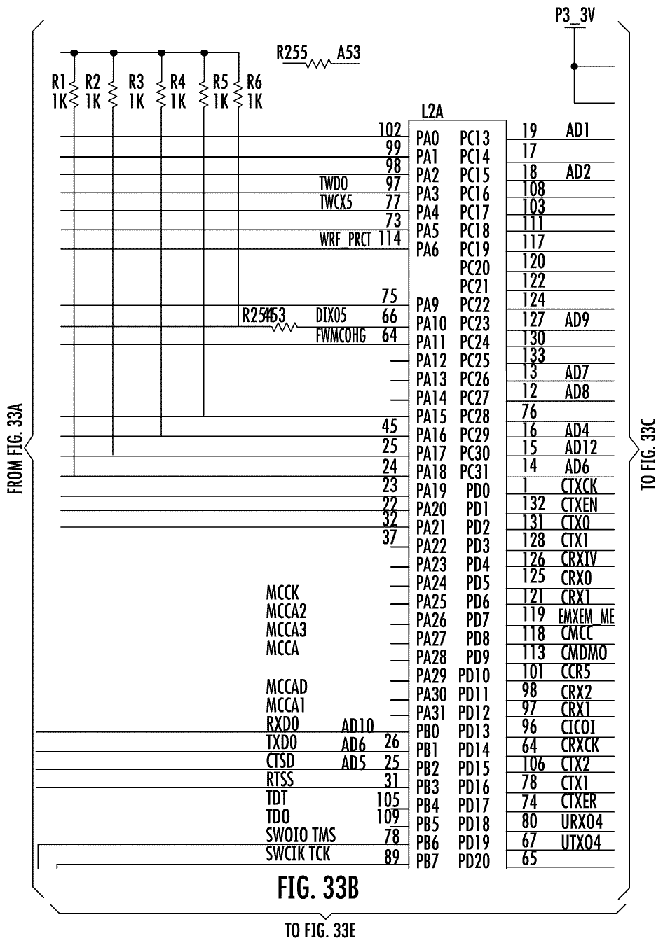

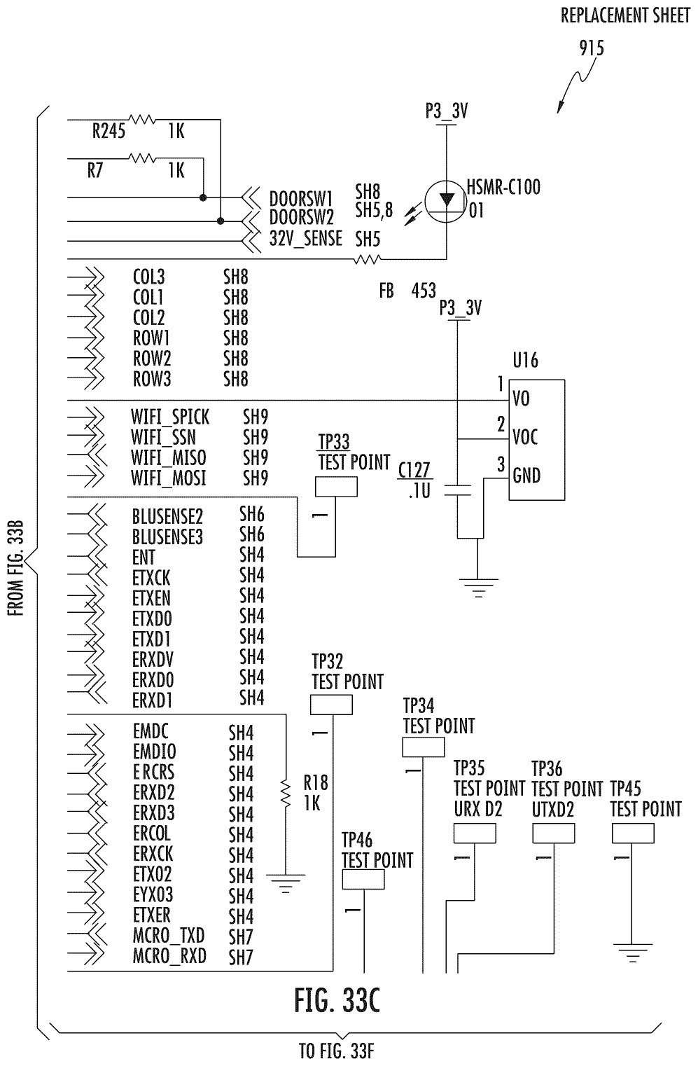

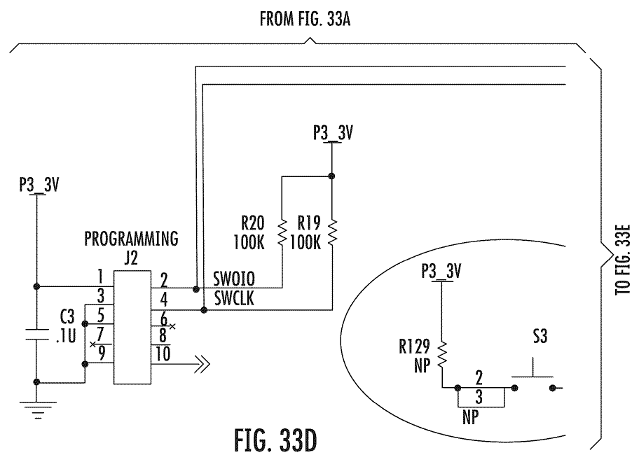

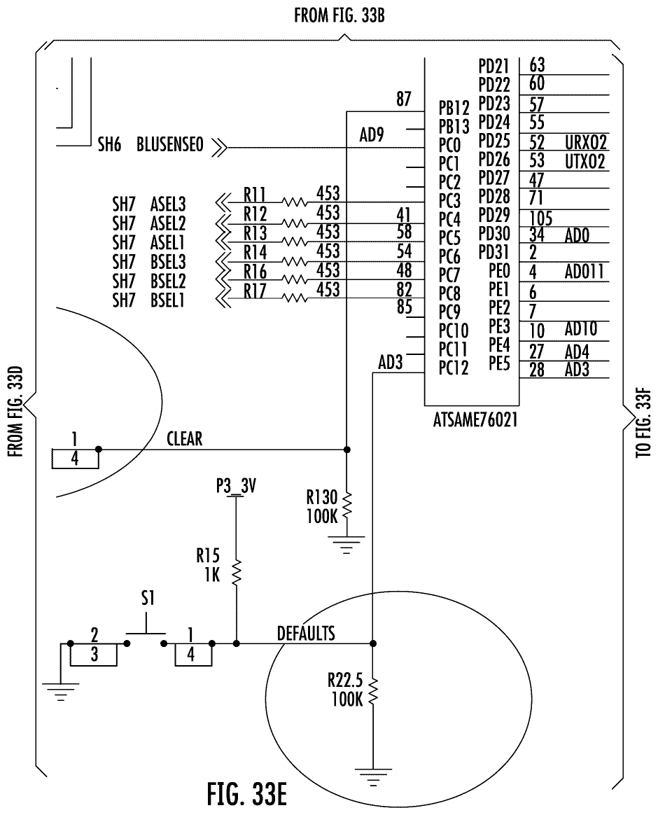

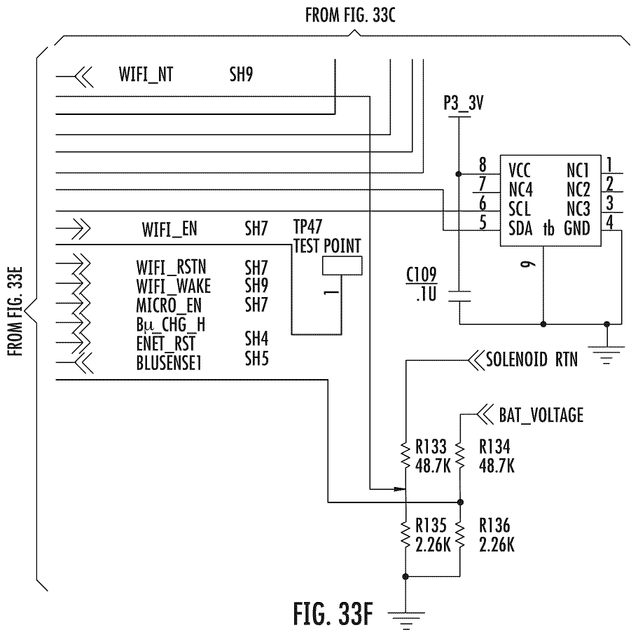

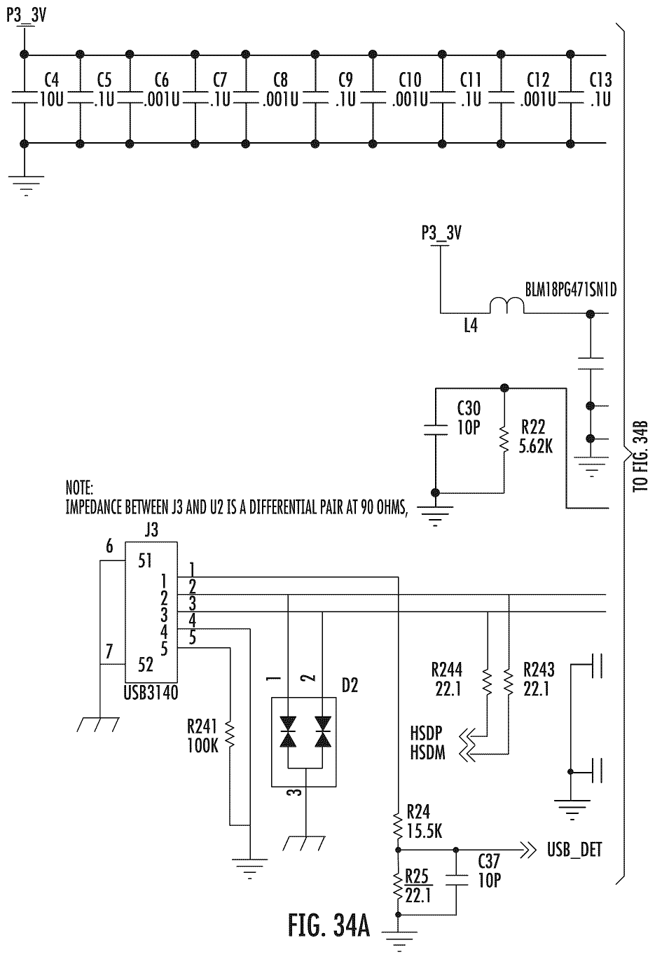

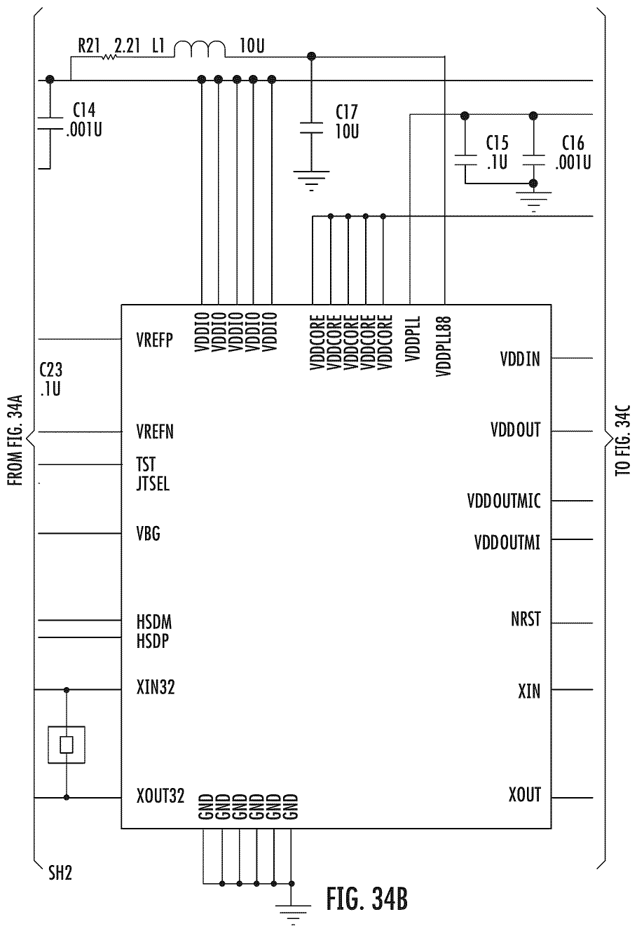

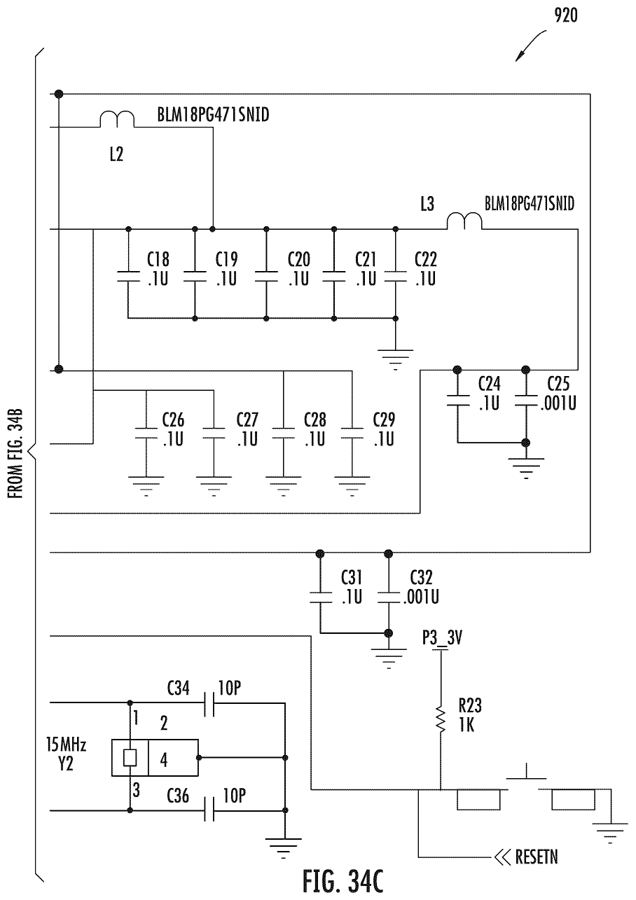

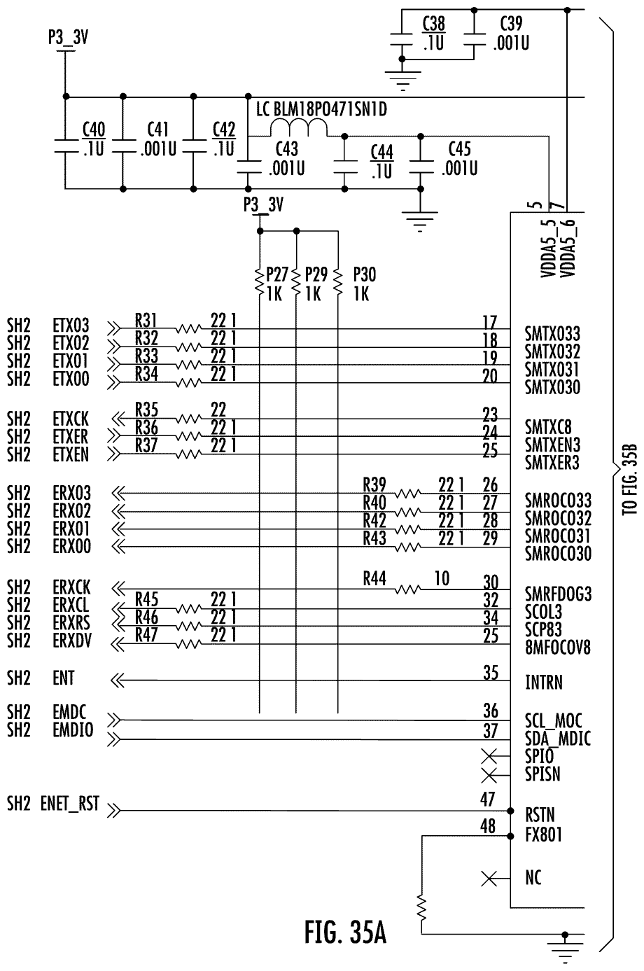

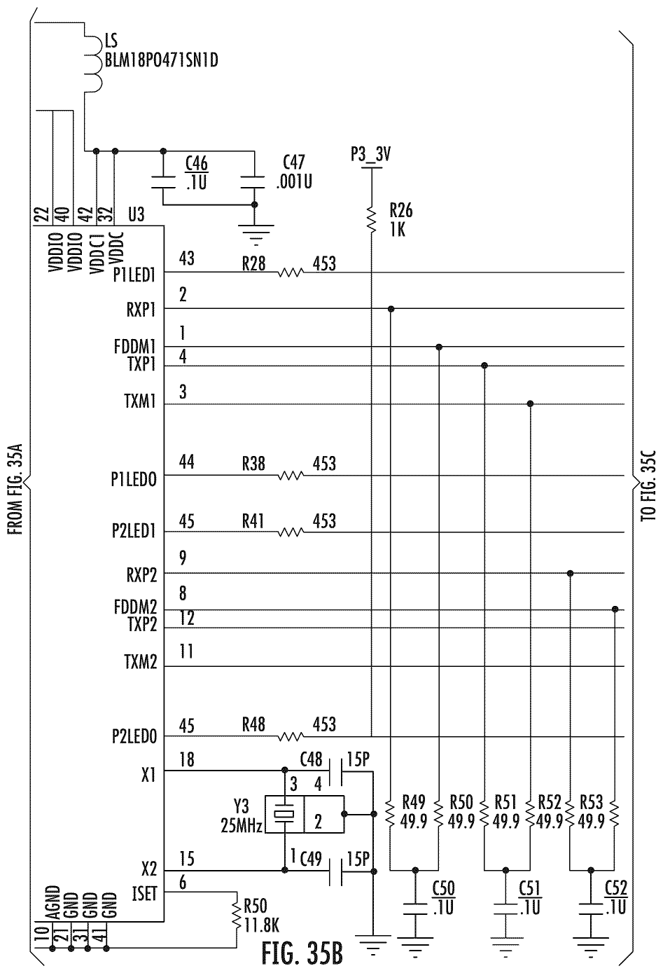

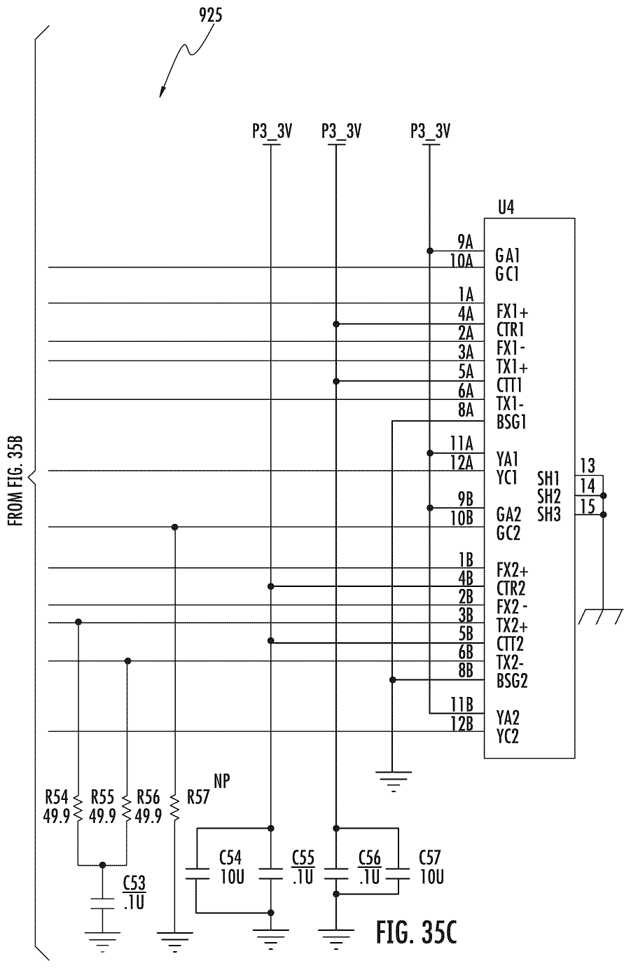

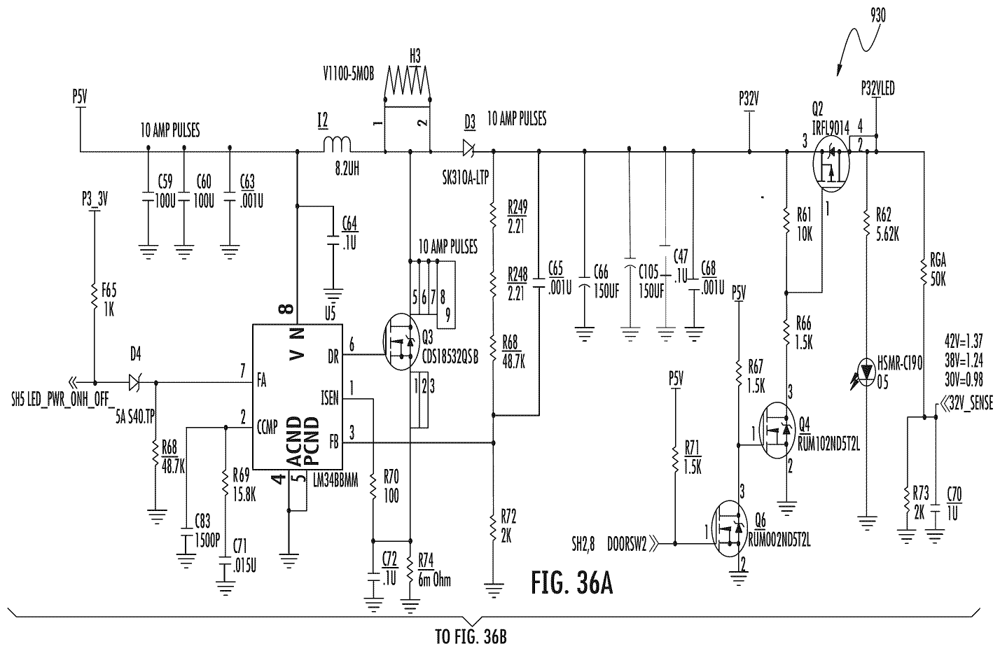

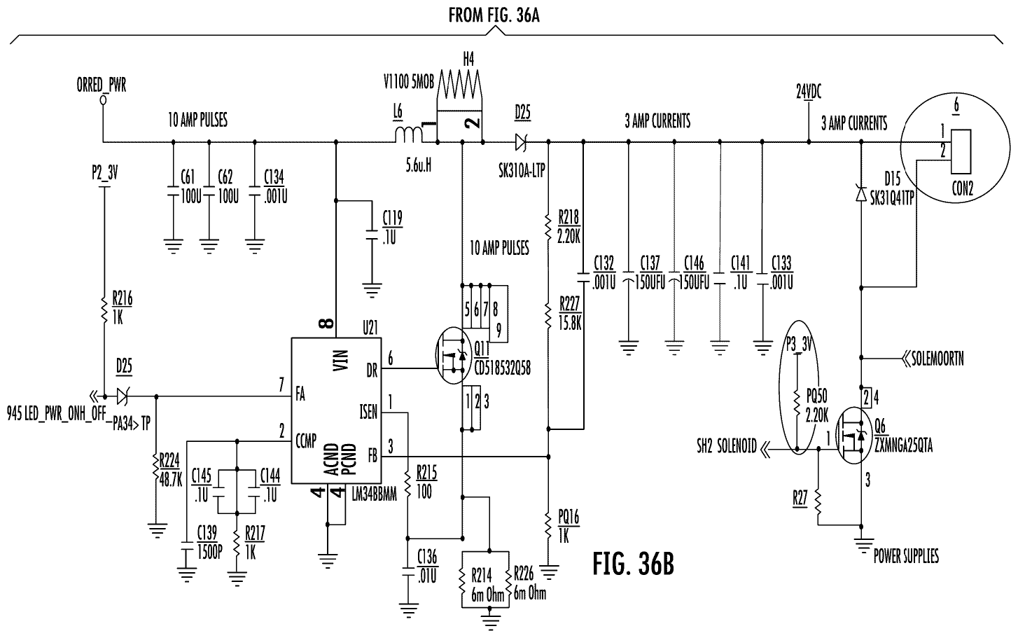

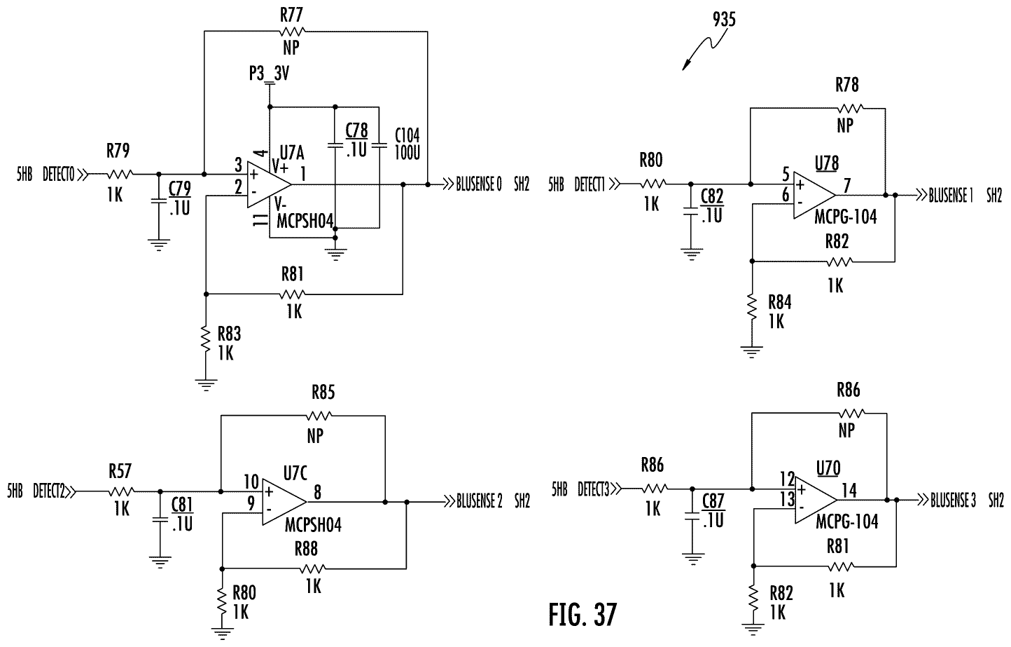

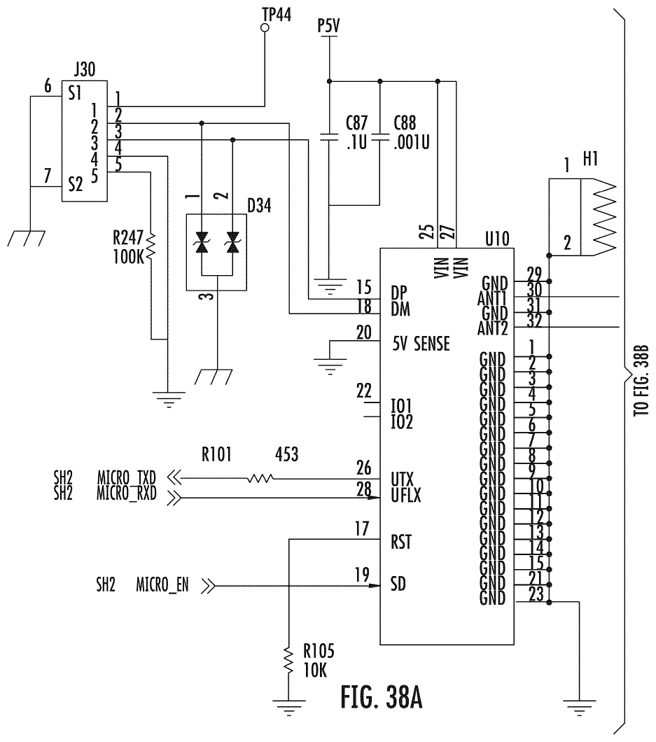

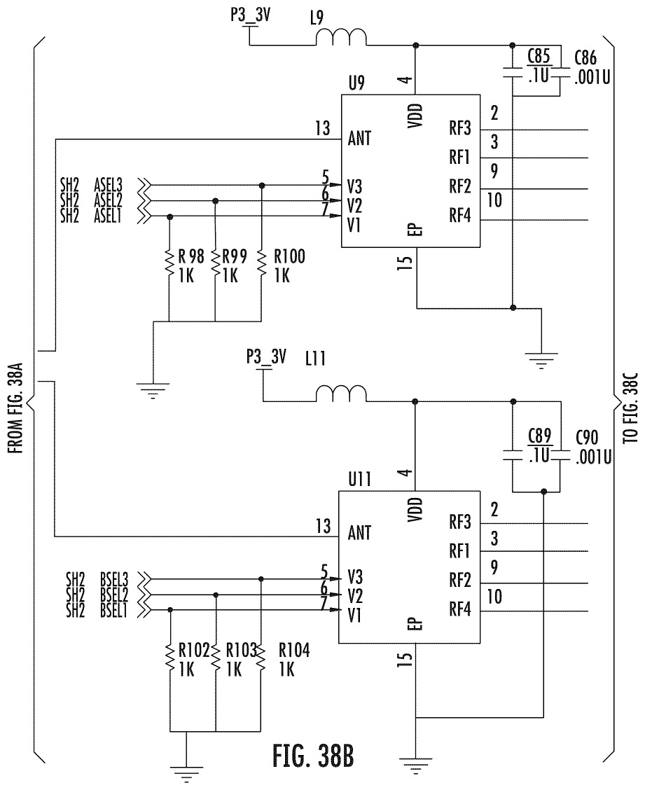

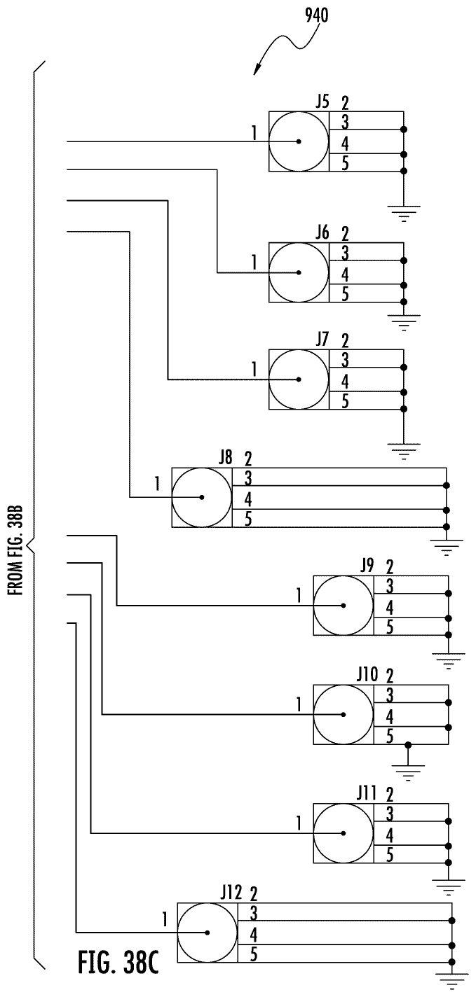

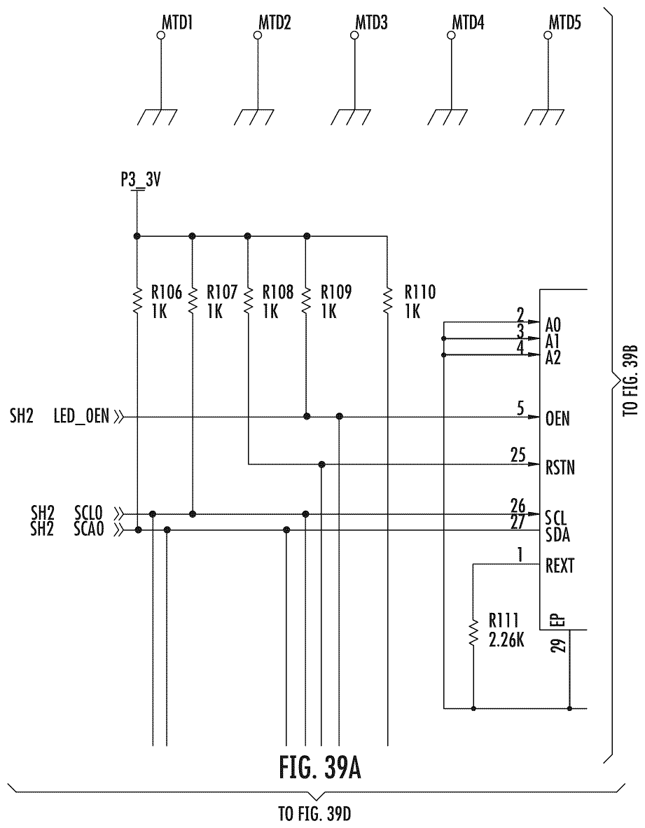

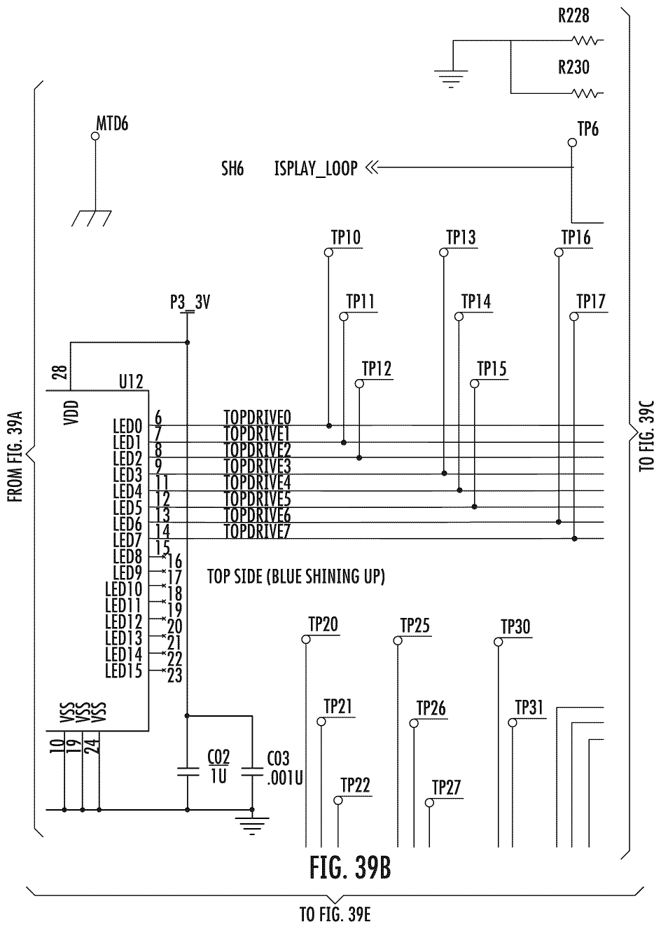

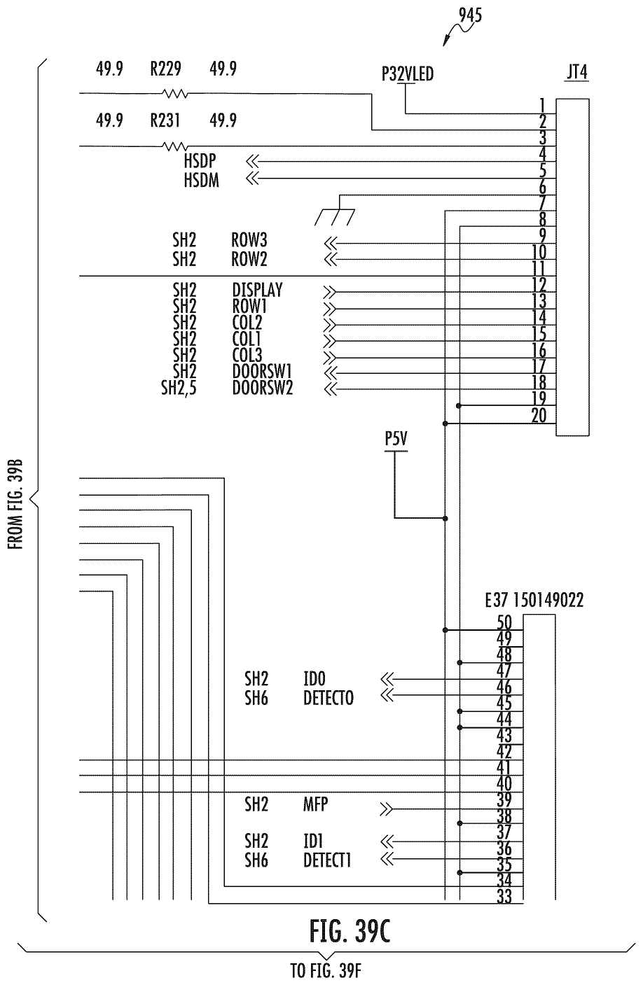

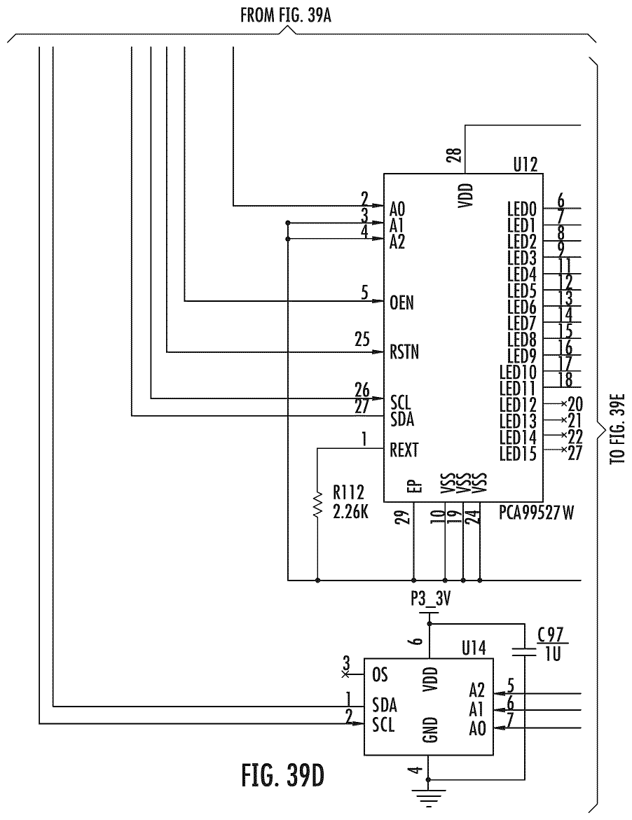

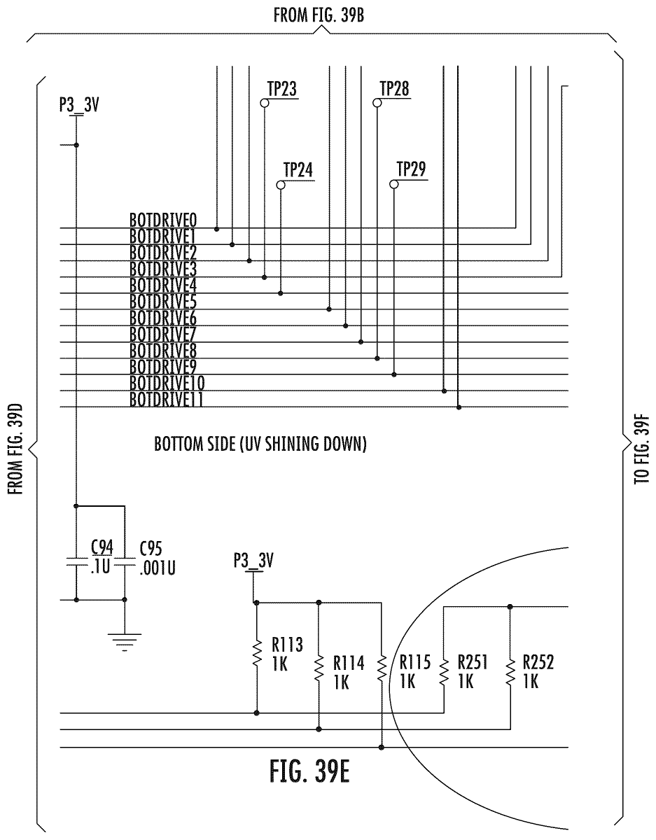

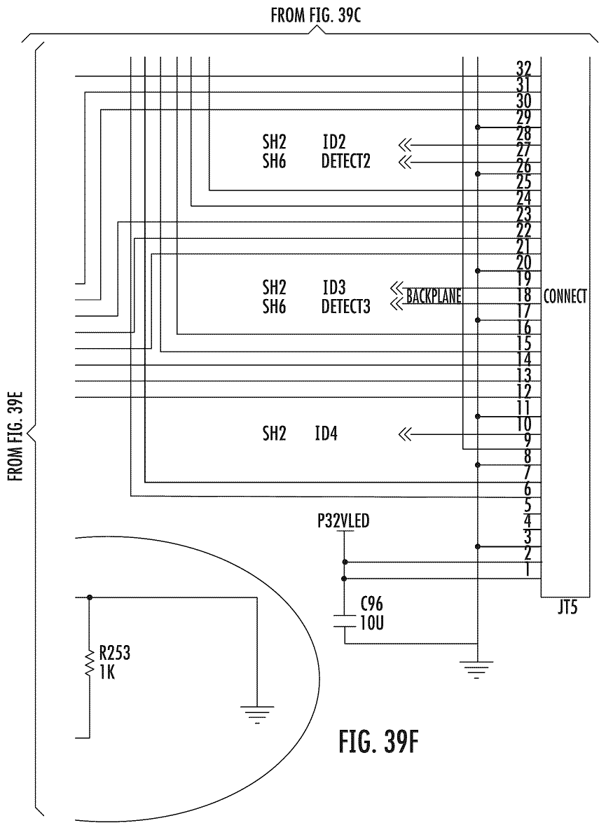

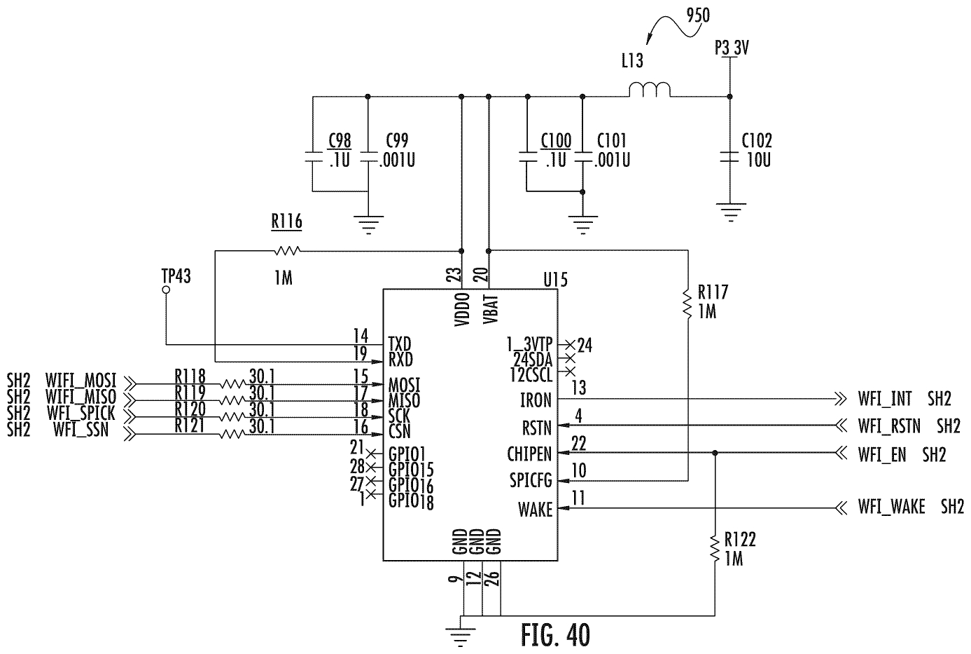

Referring now to FIGS. 31-40, circuit diagrams 905, 910, 915, 920, 925, 930, 935, 940, 945, 950 illustrate several components from the UV sterilization device 101a-101d.

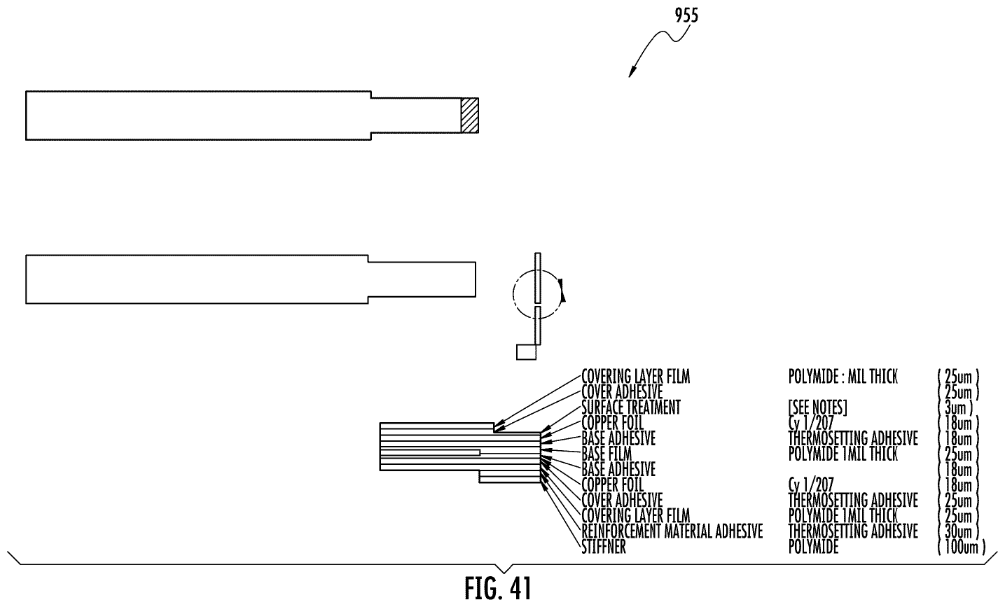

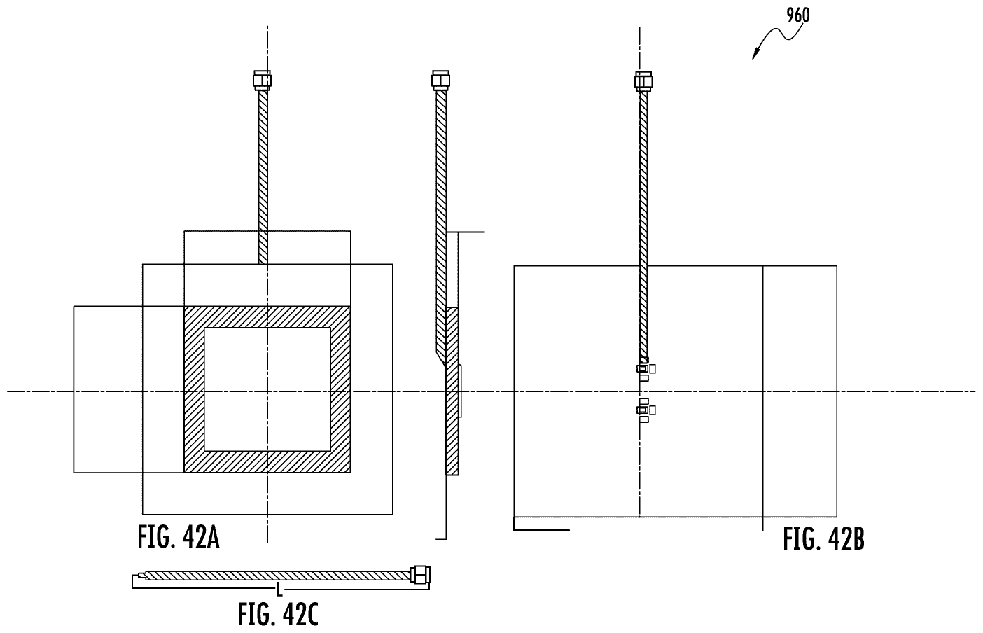

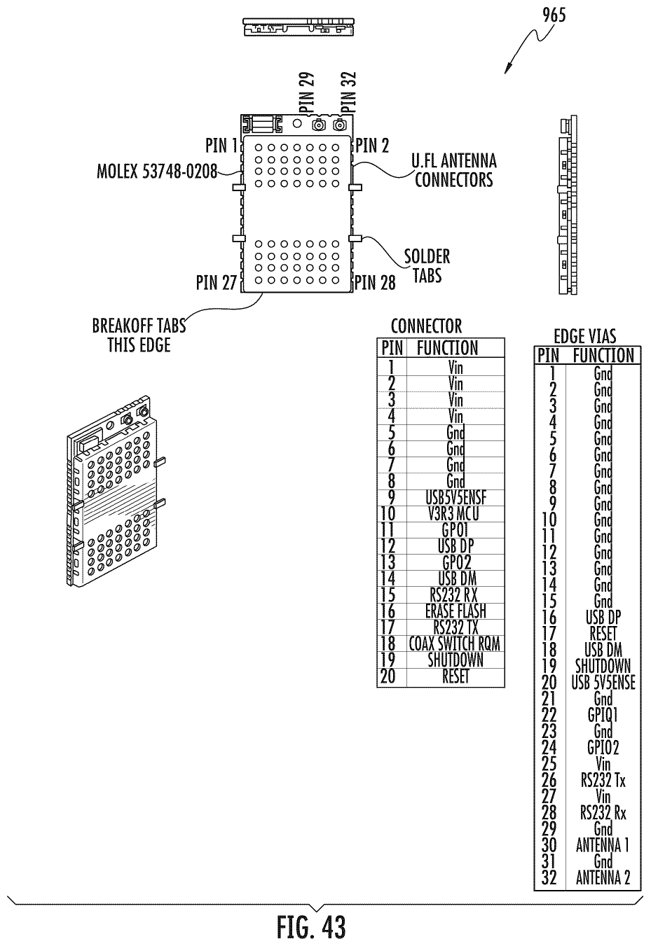

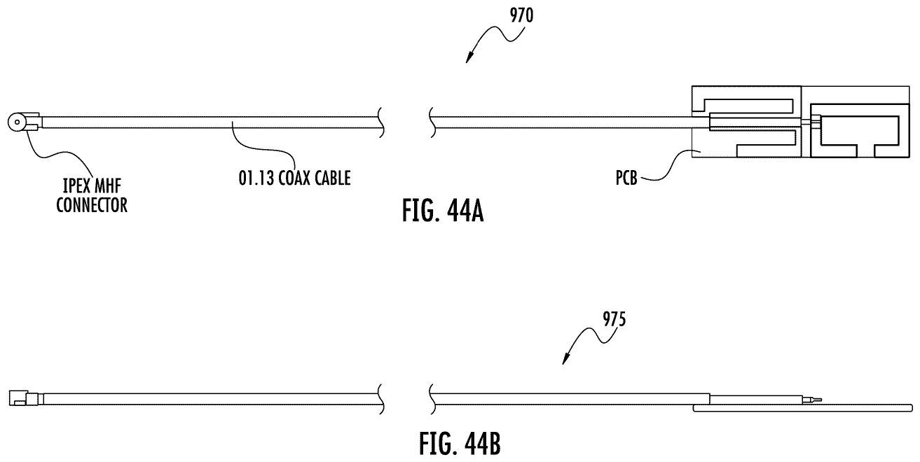

Referring now to FIGS. 41-44B, diagrams 955, 960, 965, 970, 975 illustrate several components from the UV sterilization device 101a-101d.

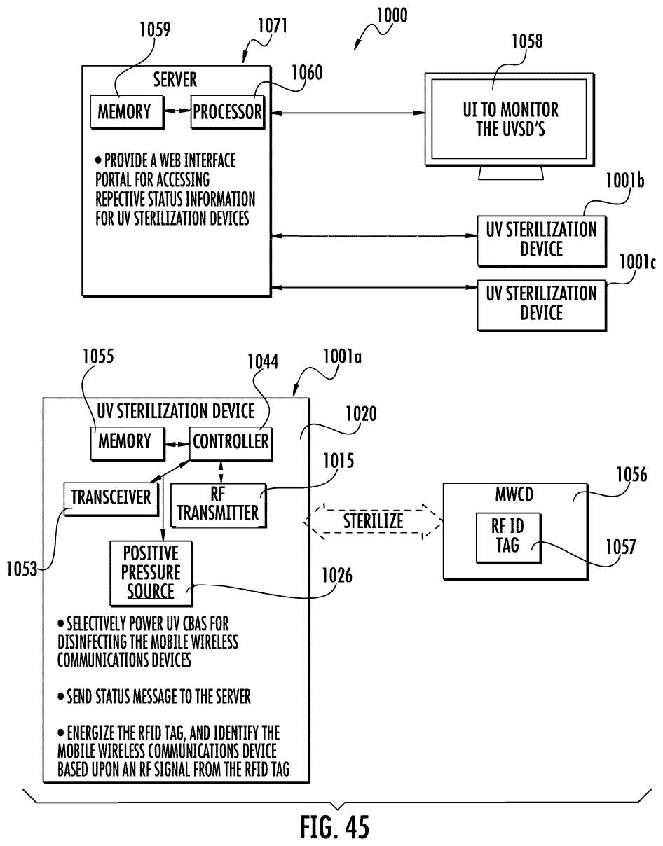

Referring now additionally to FIG. 45, another embodiment of the UV sterilization system 100 is now described. In this embodiment of the UV sterilization system 1000, those elements already discussed above with respect to FIG. 1 are incremented by 900 and most require no further discussion herein. This embodiment differs from the previous embodiment in that this UV sterilization system 1000 illustratively includes the server 1011 having a memory 1059, and a processor 1060 coupled thereto, and the UV sterilization device 1001a having a controller 1044, and a memory 1055 coupled thereto. The UV sterilization system 1000 illustratively includes a mobile wireless communications device 1056 having an RF ID tag 1057 therein, and a remote terminal 1058 communication with the server 1011.

Generally, in the UV sterilization device 101a-101d, the plurality of LED UV sources 135a-1351, 139a-139e is configured to irradiate the device with UV radiation with an emission spectrum having a spectral width less than the entire UV band. Each of the plurality of trays 131a-131d comprises a material transparent to the emission spectrum.

In particular, the emission spectrum may comprise only UV-C radiation (i.e., wavelengths greater than 280 nm are not emitted). The spectral width less than the entire UV band does not damage or degrade the materials of the device being disinfected. In typical approaches where disinfections of such devices are performed using the entire UV band (e.g., using mercury UV bulbs), there may be damage to the devices, for example, material decomposition, discoloration, crazing, surface pitting, or cracking, which can be observed through SEM imaging, spectroscopy, or other methods known to those skilled in the art. As will be appreciated, the degraded surfaces create UV shadows, preventing the surface from being fully irradiated, reducing efficacy, and rendering the device surface unsuitable for 360-degree disinfection. Further, since polymer plastic is a common housing material for electronic devices being sterilized, the premature aging of the housings will reduce the life cycle of the device for day-to-day user.

In the following, experimental data for the degradation of plastic polymers in electronic devices is now described. In the experiment, the sample plastic pieces were subjected to repeated sterilizations using a typical UV sterilization device and the UV sterilization device 101a-101d of the present disclosure.

Applicant has conducted experiments on Acrylonitrile Butadiene Styrene (ABS) plastics exposed to the entire UV band spectrum from a low-pressure mercury lamp. Visually, the ABS noticeably yellowed from UV damage. Mechanically, ABS loses significant tensile strength and elongation at break reduces by 40%, meaning the UV damage makes it more brittle. Further, evidence of damage can be observed from thermal analysis techniques. Other materials exposed to UV-C LEDs with a spectral width less than the entire UV band in the UV sterilization device 101a-101d were not damaged by their exposure at an equivalent time period. This suggests that the UV sterilization device 101a-101d is a better choice to preserve the working life of devices needing UV disinfection.

### Introduction

In prior work, Applicant irradiated materials from Vocera badges with UV-C light generated by LEDs in the UV sterilization device 101a-101d, and subsequently tested those materials with a thermogravimetric analyzer (TGA). The Vocera materials remained unaffected from 3 days (˜72 hours) continuous UVC LED exposure within the UV sterilization device 101a-101d, and there was no visual difference (such as discoloration) observed either.

### Results

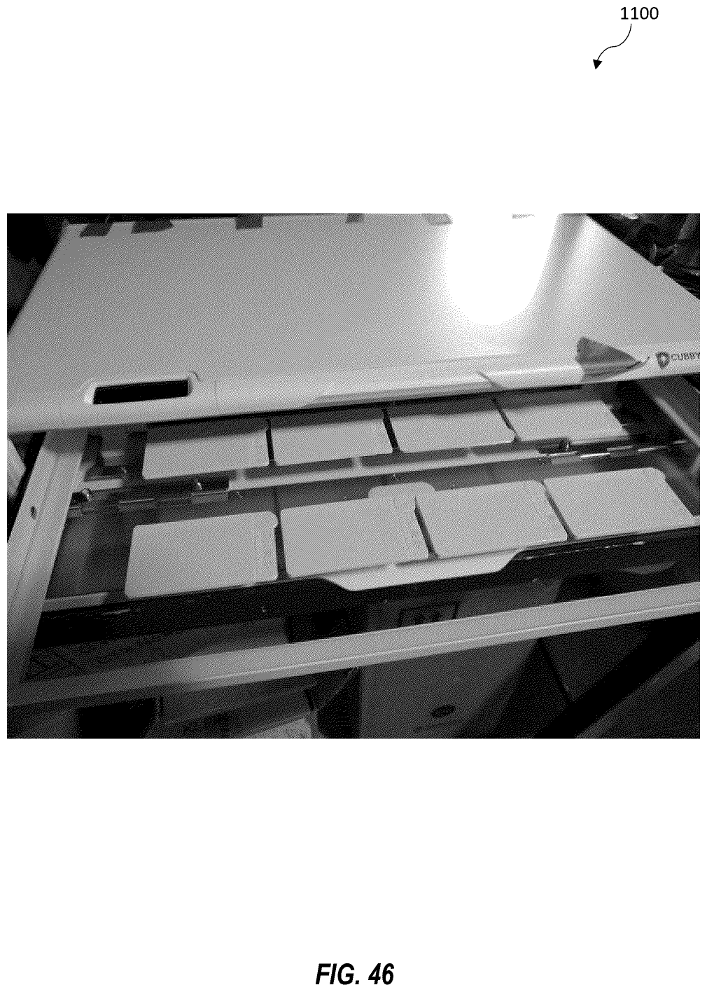

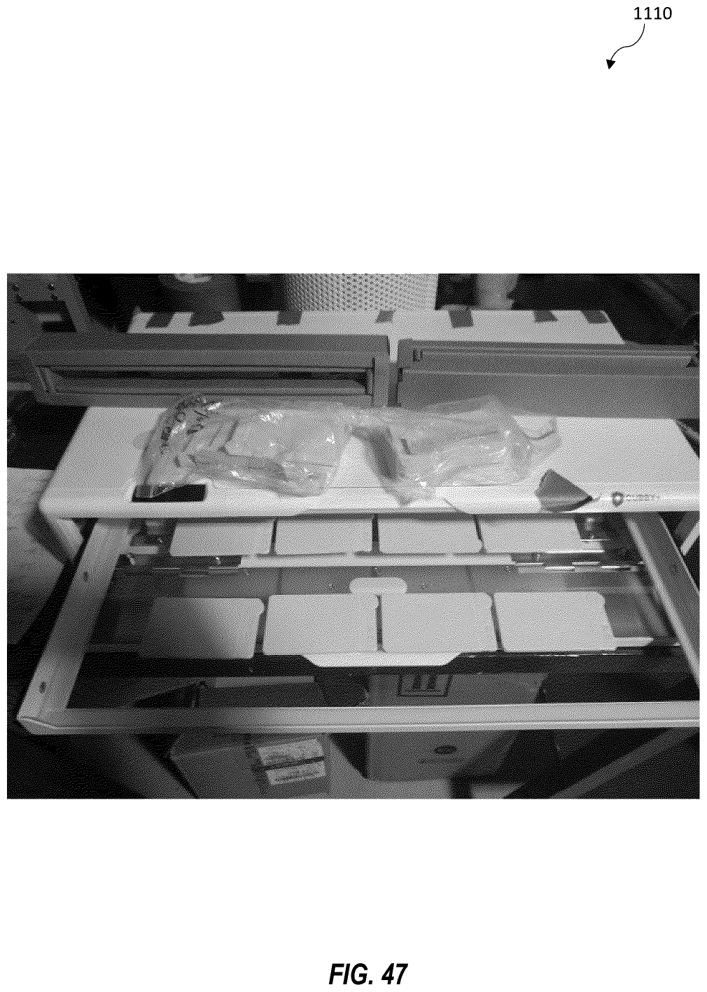

This study expanded upon the prior work by examining the effects of UV exposure from a traditional low-pressure mercury lamp (LPML), which emits across the entire UV band spectrum, on ABS plastic plaques through tensile testing and thermogravimetric analysis. The plaques were continuously exposed for at least 3 days to the entire UV band spectrum in a Vioguard Cubby system with four LPMLs to match the time from the prior work. Visual inspection was performed by Applicant. Tensile testing was performed at Briem Engineering using an Instron mechanical test unit. SEM images were taken at Briem Engineering's optical lab. TGA testing was performed using a TA Instruments 5500 unit. Referring now to FIG. 46, an image 1100 shows fresh ABS plaques with no UV exposure. Referring now to FIG. 47, an image 1110 shows yellowed ABS plaques after being exposed to entire UV band radiation for three days. Visually, the samples exposed to the entire UV spectrum light from the LPMLs were substantially yellowed and darkened. This initial detail immediately confirms material degradation on its own. Tensile testing showed a loss of at least 10% of normal mechanical strength and elongation at break reduced by 40%, presented below in Table 1.

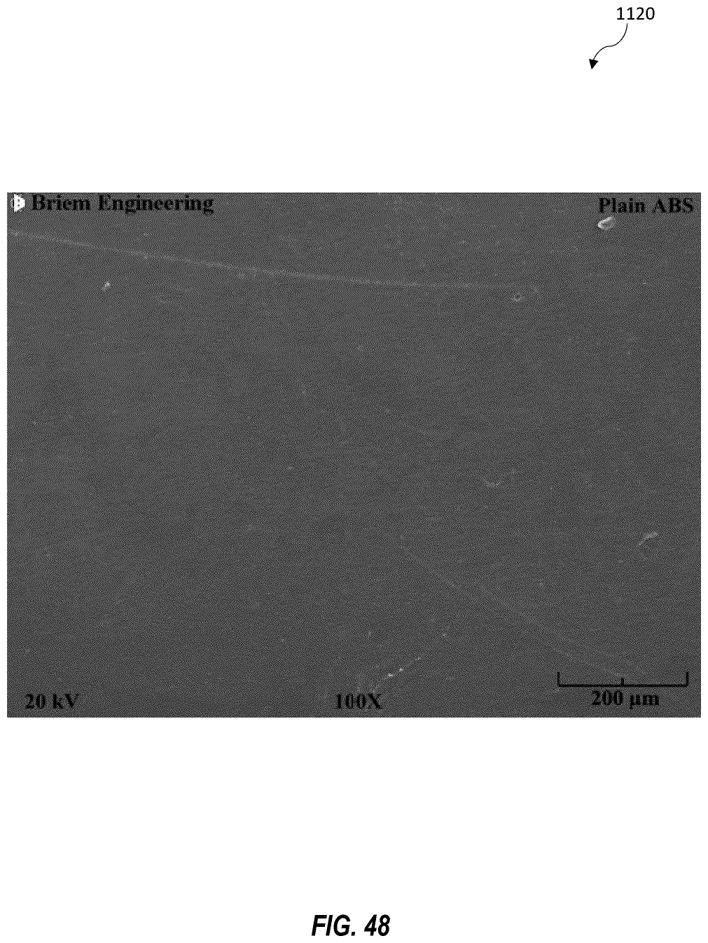

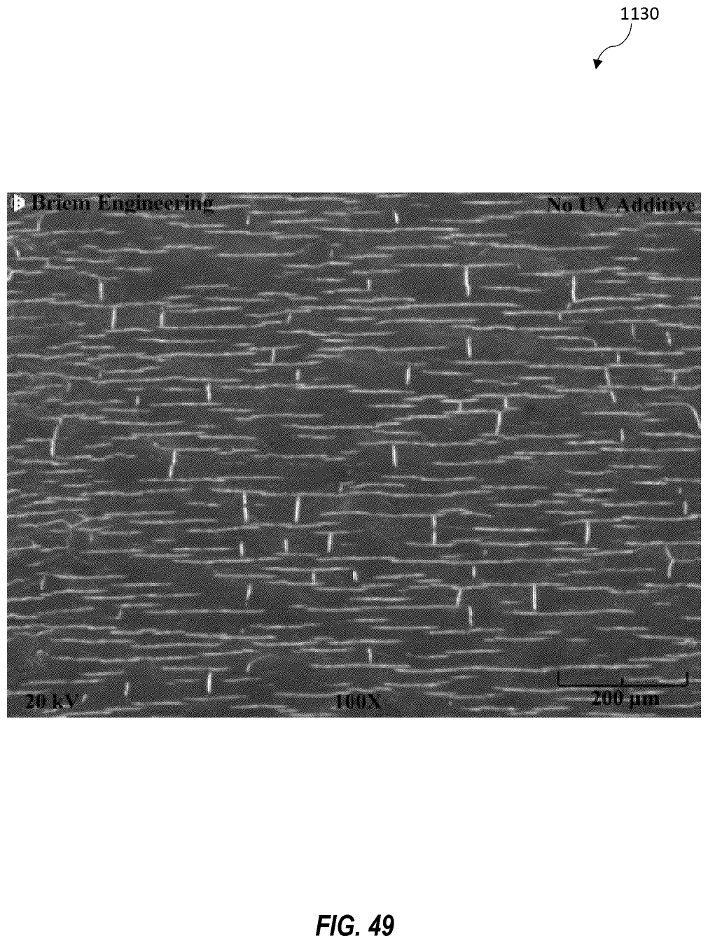

Referring now additionally to FIGS. 48-49, Briem Engineering also obtained SEM images 1120 (before entire band UV exposure), 1130 (after entire band UV exposure) of the surfaces of the UV exposed and non-exposed ABS plaques. The results were rather dramatic: while non-exposed ABS of course displayed plain, smooth results, ABS exposed to the mercury lamps clearly formed a complex network of microfissures. These microfissures appear as relatively long “canyons”, with length scales anywhere from 20-200+microns in length and 1-10 microns in width. Even the relatively small aspect is large enough that pathogenic microorganisms could become lodged in the fissure and form biofilm and colonies. This would make devices containing ABS (or other similar polymers) very difficult to be disinfected by UV (since the radiation cannot reach the microorganism as easily), and thus become a more significant long-term threat for infection transmission as more degradation occurs.

The TGA results showed a substantial difference in the weight loss profile for the ABS plaques exposed to UV-C. Comparing these new TGA results to the prior work, the materials exposed to the UV-C LEDs showed far less effects from UV degradation (virtually none) compared to those materials exposed to LPMLs for the same exposure time. While the absolute dosages were different, devices in the field disinfected by these UV sources will tend to spend roughly the same amount of time within them to achieve sufficient efficacy.

### Tensile Testing Results

The base ABS resin had a tensile strength of 6.45 psi. Following three days of entire UV band exposure, the damaged plaque had a tensile strength of 5.86 psi, a loss of ˜10% mechanical strength. Elongation at break also reduced by 40%.

TABLE 1
No UV Exposure
ABS Plain Tensile Results:

| | | | | % | Max | Tensile |
|---|---|---|---|---|---|---|
| | Width, | Thickness, | Area, | Elongation, | Load, | Strength, |
| Item | in | In | in² | 2 in | lbs. | psi |
| 1 | 0.5110 | 0.1045 | 0.0534 | 17.0 | 345 | 6,461 |
| 2 | 0.4700 | 0.1040 | 0.0489 | 8.0 | 315 | 6,442 |
| 3 | 0,4525 | 0.1045 | 0.0473 | 9.5 | 307 | 6,490 |
| 4 | 0.4600 | 0.1040 | 0.0478 | 5.5 | 306 | 6,402 |
| 5 | 0.4720 | 0.1045 | 0.0493 | 7.5 | 319 | 6,471 |
| Avg. | | | | 9.5 | | 6,453 |

TABLE 2
Following Three Days Entire UV Band Exposure
ABS Plain Tensile Results:

| | | | | % | Max | Tensile |
|---|---|---|---|---|---|---|
| | Width, | Thickness, | Area, | Elongation, | Load, | Strength, |
| Item | in | In | in² | 2 in | lbs. | psi |
| 1 | 0.5085 | 0.10285 | 0.0523 | 3.5 | 288 | 5,507 |
| 2 | 0.4995 | 0.10450 | 0.0522 | 4.0 | 288 | 5,517 |
| 3 | 0.5015 | 0.10380 | 0.0520 | 5.5 | 308 | 5,923 |
| 4 | 0.4905 | 0.10410 | 0.0511 | 6.5 | 314 | 6,145 |
| 5 | 0.5085 | 0.10250 | 0.0521 | 6.5 | 325 | 6,238 |
| Avg. | | | | 5.2 | 305 | 5,866 |

### TGA Results

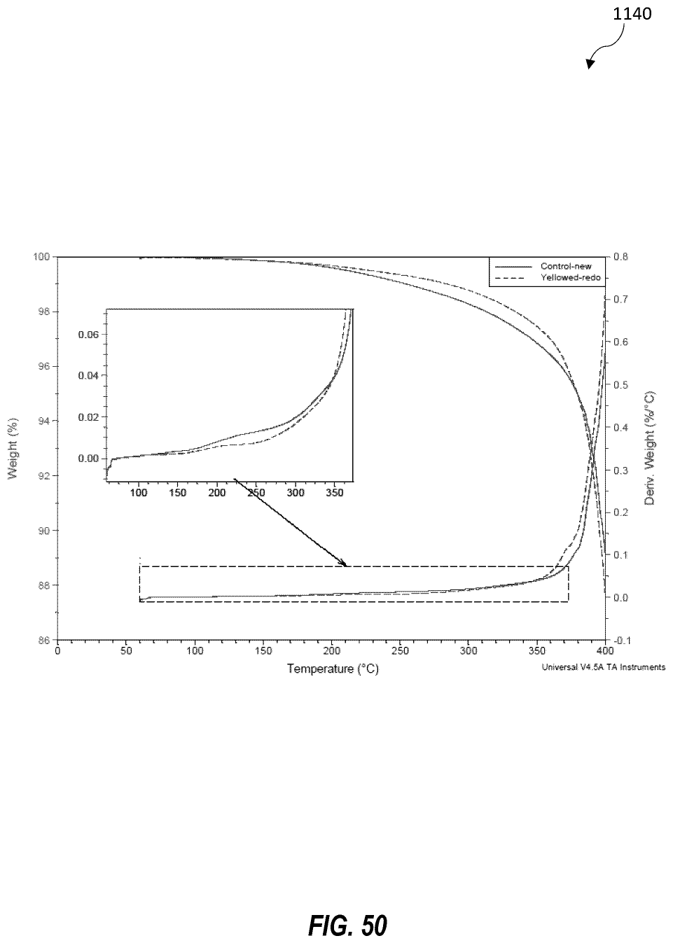

Referring now to FIG. 50, a diagram 1140 is now described. The UV damaged “Yellowed” sample deviated from the control in both the Weight loss (%) profile and in the derivative weight (%/C). This suggests that there is a substantial difference the material structure, evidenced most in the 200-250° C. range where there is greatest first derivative deviation. This might be attributed to PAN cyclization initiated by UV energy, but more advanced testing would be required to determine if this is exactly the case.

Comparison TGA Results from Vocera Badges

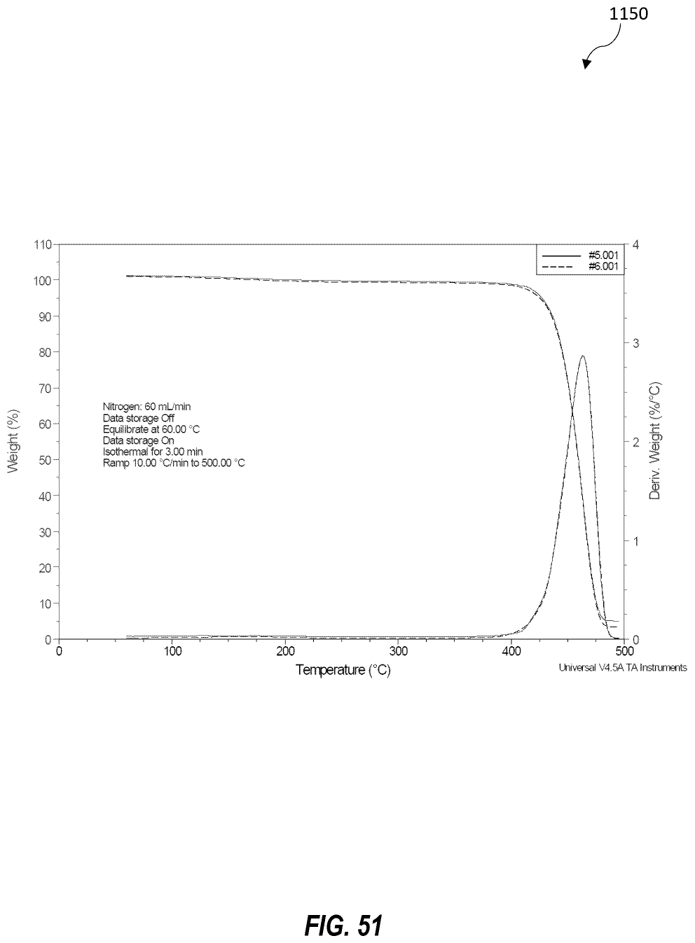

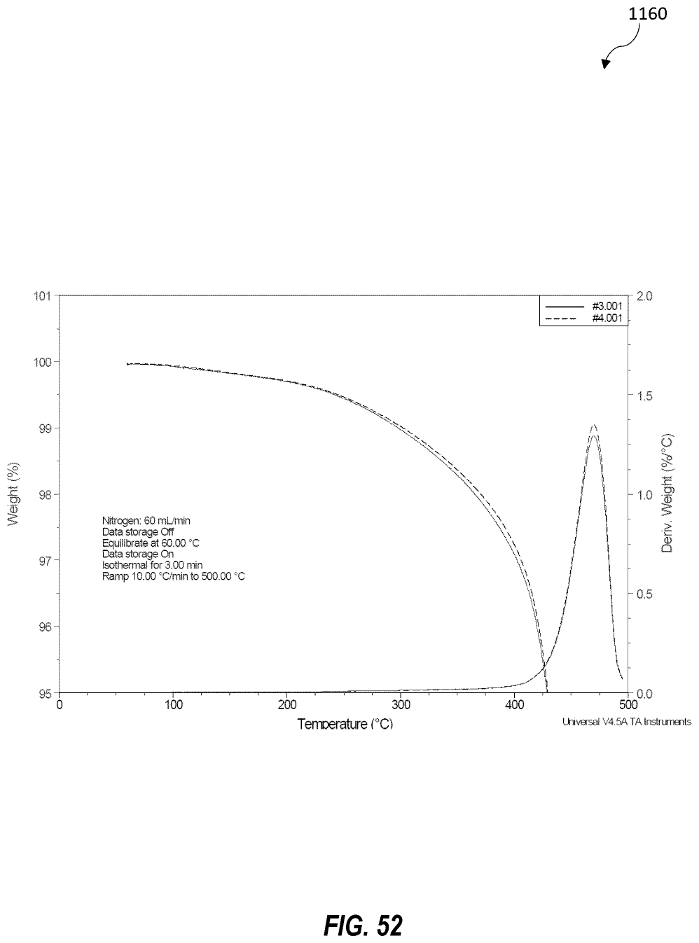

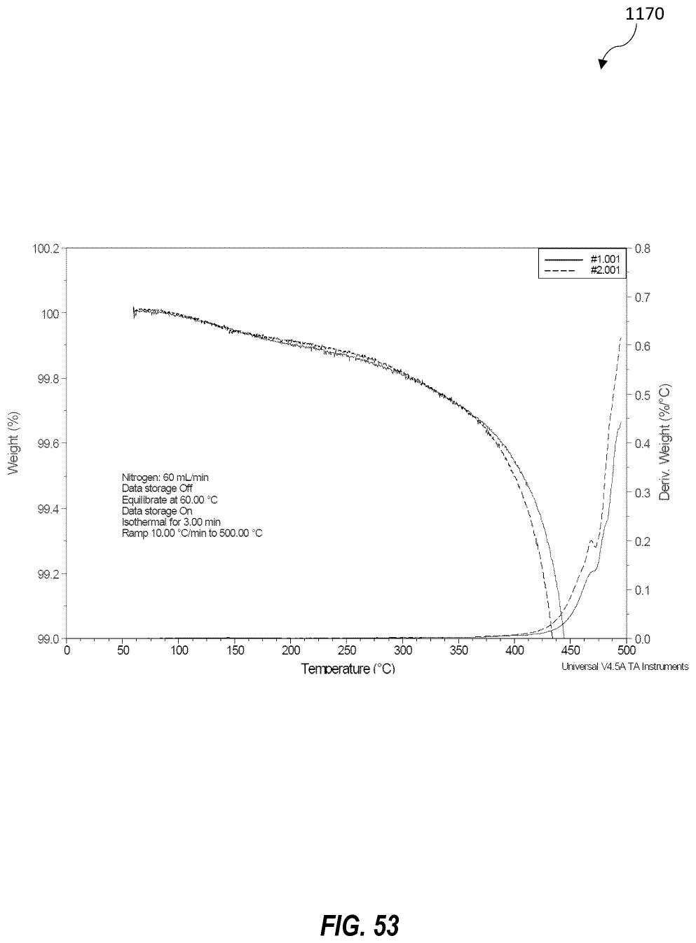

Referring now to FIG. 51, a diagram 1150 is now described and shows a comparison of TGA curves for less than the entire UV band exposed PA12 and PA12 control. Referring now to FIG. 52, a diagram 1160 is now described and shows a comparison of TGA curves for UV-exposed TPU-Silicone and TPU-Silicone control. Referring now to FIG. 53, a diagram 1170 is now described, which shows a comparison of TGA curves for less than the entire UV band exposed PA12 and PA12 control.

Many modifications and other embodiments of the present disclosure will come to the mind of one skilled in the art having the benefit of the teachings presented in the foregoing descriptions and the associated drawings. Therefore, it is understood that the present disclosure is not to be limited to the specific embodiments disclosed, and that modifications and embodiments are intended to be included within the scope of the appended claims.

1. An ultraviolet (UV) sterilization device comprising:
a housing defining a cavity therein, and having a door configured to permit access to the cavity;
at least one tray carried within the cavity and configured to receive at least one device;
at least one UV circuit board assembly (CBA) carried within the cavity adjacent the at least one tray and comprising a circuit board, and a plurality of light emitting diode ultraviolet (LED UV) sources carried by the circuit board and configured to irradiate the at least one device with UV radiation with an emission spectrum having a spectral width less than the entire UV band, the at least one tray comprising a material transparent to the emission spectrum; and
a controller coupled to the at least one UV CBA and configured to selectively power the at least one UV CBA for disinfecting the at least one device.

2. The UV sterilization device of claim 1 wherein the plurality of LED UV sources is configured to perform 360 degree irradiation of the at least one device without UV shadowing.

3. The UV sterilization device of claim 1 further comprising a positive air pressure source carried by the housing and configured to create positive air pressure in the cavity when the door is open.

4. The UV sterilization device of claim 3 further comprising an air filter coupled to the positive air pressure source.

5. The UV sterilization device of claim 1 wherein the controller is configured to drive the plurality of LED UV sources with a stepped waveform.

6. The UV sterilization device of claim 1 wherein the controller is configured to monitor duty cycle of the plurality of LED UV sources.

7. The UV sterilization device of claim 1 wherein the material comprises quartz.

8. The UV sterilization device of claim 1 wherein the controller is configured to selectively power the at least one UV CBA when the at least one device is detected on a respective tray.

9. The UV sterilization device of claim 1 further comprising a keypad carried on an external surface of the housing and coupled to the controller; and wherein the controller is configured to unlock the door based upon a code received from the keypad.

10. The UV sterilization device of claim 1 further comprising a transceiver configured to communicate with a server; and wherein the transceiver comprises a wireless transceiver coupled to the controller and configured to communicate with the server via a wireless base station.

11. The UV sterilization device of claim 1 further comprising a near field communications (NFC) device configured to communicate with the at least one device when external to the cavity and unlock the door when the at least one device is authorized.

12. An ultraviolet (UV) sterilization device comprising:
a housing defining a cavity therein, and having a door configured to permit access to the cavity;
at least one tray carried within the cavity and configured to receive at least one device;
at least one UV circuit board assembly (CBA) carried within the cavity adjacent the at least one tray and comprising a circuit board, and a plurality of light emitting diode ultraviolet (LED UV) sources carried by the circuit board and configured to
irradiate the at least one device with UV radiation with an emission spectrum having a spectral width less than the entire UV band, and
perform 360 degree irradiation of the at least one device without UV shadowing, the at least one tray comprising a material transparent to the emission spectrum;
a positive air pressure source carried by the housing and configured to create positive air pressure in the cavity when the door is open;
a wireless transceiver configured to communicate with a server; and
a controller coupled to the at least one UV CBA and the wireless transceiver, the controller configured to selectively power the at least one UV CBA for disinfecting the at least one device, and communicate with the server via a wireless base station.

13. The UV sterilization device of claim 12 further comprising an air filter coupled to the positive air pressure source.

14. The UV sterilization device of claim 12 wherein the controller is configured to drive the plurality of LED UV sources with a stepped waveform.

15. The UV sterilization device of claim 12 wherein the controller is configured to monitor duty cycle of the plurality of LED UV sources.

16. The UV sterilization device of claim 12 wherein the material comprises quartz.

17. The UV sterilization device of claim 12 wherein the controller is configured to selectively power the at least one UV CBA when the at least one device is detected on a respective tray.

18. A method of making an ultraviolet (UV) sterilization device, the method comprising:
forming a housing defining a cavity therein, and having a door configured to permit access to the cavity;
positioning at least one tray to be carried within the cavity and configured to receive at least one device;
positioning at least one UV circuit board assembly (CBA) to be carried within the cavity adjacent the at least one tray and comprising a circuit board, and a plurality of light emitting diode ultraviolet (LED UV) sources to be carried by the circuit board and configured to irradiate the at least one device with UV radiation with an emission spectrum having a spectral width less than the entire UV band, the at least one tray comprising a material transparent to the emission spectrum; and
coupling a controller to the at least one UV CBA, the controller configured to selectively power the at least one UV CBA for disinfecting the at least one device.

19. The method of claim 18 wherein the plurality of LED UV sources is configured to perform 360 degree irradiation of the at least one device without UV shadowing.

20. The method of claim 18 further comprising positioning a positive air pressure source to be carried by the housing, the positive air pressure source configured to create positive air pressure in the cavity when the door is open.
